(19) United States
(12) Patent Application Publication
(10) Pub. No.: US 2023/0227566 A1
(43) Pub. Date: Jul. 20, 2023

(54) AMHRII-BINDING COMPOUNDS FOR PREVENTING OR TREATING LUNG CANCERS

(71) Applicants: GAMAMABS PHARMA, Toulouse (FR); INSTITUT CURIE, Paris Cedex 05 (FR)

(72) Inventors: Jean-Marc BARRET, Castres (FR); Jean-François PROST, Versailles (FR); Mehdi LAHMAR, Viroflay (FR); Stéphane DEGOVE, Pantin (FR); Olivier DUBREUIL, Mauressac (FR); André NICOLAS, Paris (FR); Didier MESEURE, Paris (FR)

(21) Appl. No.: 16/605,053

(22) PCT Filed: Apr. 13, 2018
(86) PCT No.: PCT/EP2018/059553

§ 371 Date: Oct. 14, 2019

(30) Foreign Application Priority Data
Apr. 14, 2017 (EP) 17305446.1

(51) Int. Cl. C07K 16/28; A61K 35/17; A61K 35/15; A61K 38/17; A61K 47/68; A61P 35/00; A61P 11/00

(52) U.S. Cl. CPC C07K 16/2869; A61K 35/17; A61K 35/15; A61K 38/1774; A61K 47/6849; A61P 35/00; A61P 11/00; A61K 2039/505

(57) ABSTRACT

The present invention relates to a human AMHRII-binding agent for its use for preventing or treating a lung cancer, and especially a non-small call lung cancer (NSCLC), and even more especially a NSCLC selected in a group comprising epidermoid NSCLC, adenocarcinoma NSCLC, large cells NSCLC and squamous cell carcinoma NSCLC and neuroendocrine NSCLC.

### FIELD OF THE INVENTION

The present invention relates to the field of lung cancer treatment.

### BACKGROUND OF THE INVENTION

Lung cancer is the malignant transformation and expansion of lung tissue, and is responsible for 1.3 million deaths worldwide annually. It is the most common cause of cancer-related death in men, and the second most common in women.

The World Health Organization classifies lung cancer into four major histological types: (1) squamous cell carcinoma (SCC), (2) adenocarcinoma, (3) large cell carcinoma, and (4) small cell lung carcinoma (SCLC). The term non-small cell lung carcinoma (NSCLC) includes squamous, adenocarcinoma and large cell carcinomas.

The non-small cell lung cancers (NSCLC) are grouped together because their prognosis and management are roughly identical. There are three main sub-types: squamous cell lung carcinoma, adenocarcinoma and large cell lung carcinoma. Squamous cell carcinoma, accounting for 29% of lung cancers, also starts in the larger bronchi but grows slower. The size of these tumors varies on diagnosis. Adenocarcinoma is the most common subtype of NSCLC, accounting for 32% of lung cancers. It is a form which starts near the gas-exchanging surface of the lung. Most cases of adenocarcinoma are associated with smoking. However, among people who have never smoked (“never-smokers”), adenocarcinoma is the most common form of lung cancer. A subtype of adenocarcinoma, the bronchioloalveolar carcinoma, is more common in female never-smokers, and may have different responses to treatment. Other subtypes of NSCLC are neuroendocrine lung tumors (NE), acinar-type lung cancer (AT), and large cell carcinoma, a fast-growing form, accounting for 9% of lung cancers that grows near the surface of the lung.

Small cell lung cancer (SCLC, also called “oat cell carcinoma”) is a less common form of lung cancer. It tends to start in the larger breathing tubes and grows rapidly becoming quite large. The oncogene most commonly involved is L-myc. The “oat” cell contains dense neurosecretory granules which give this an endocrine/paraneoplastic syndrome association. It is initially more sensitive to chemotherapy, but ultimately carries a worse prognosis and is often metastatic at presentation. This type of lung cancer is strongly associated with smoking.

Other types of lung cancers include carcinoid, adenoid cystic carcinoma (cylindroma) and mucoepidermoid carcinoma.

Early detection is difficult since clinical symptoms are often not seen until the disease has reached an advanced stage. Currently, diagnosis is aided by the use of chest x-rays, analysis of the type of cells contained in sputum and fiberoptic examination of the bronchial passages. Treatment regimens are determined by the type and stage of the cancer, and include surgery, radiation therapy and/or chemotherapy. In spite of considerable research into therapies for the disease, lung cancer remains difficult to treat.

Known lung cancer treatments include surgery, chemotherapy, radiation therapy and targeted drug therapy.

Targeted therapy and especially targeted immunotherapy has the potential to benefit lung cancer patients for whom more conventional chemotherapy or radiation treatments are ineffective. Targeted immunotherapy includes the use of monoclonal antibodies.

The monoclonal antibodies bevacizumab (an anti-VEGF antibody) and ramucirumab (an anti-VEGFR2 antibody) are aimed at preventing tumors from producing new blood vessels, while necitumumab (an anti-EGFR) targets growth through preventing the action of another growth factor. Currently, there are at least two immune checkpoint inhibitors (Pembrolizumab/anti-PD1 and Nivolumab/anti-PD1) targeted antibodies approved for lung cancer patients. Today, long-lasting remissions and longer survival rates may be obtained with such immune-based treatments such as checkpoint inhibitors, monoclonal antibodies, therapeutic vaccines, and adoptive cell therapy.

However, there still remains a need in the art for further tools for the therapy of lung cancers, that may be alternative or complementary of the existing therapies.

### SUMMARY OF THE INVENTION

This invention relates to a human AMHRII-binding agent for its use for preventing or treating a lung cancer. Then, this invention relates to a human AMHRII-binding agent for use in a method of preventing or treating a lung cancer in a patient affected with a lung cancer.

Lung cancer may be selected in a group comprising non-small cell lung cancer (NSCLC) and especially a NSCLC selected in a group comprising epidermoid NSCLC, adenocarcinoma NSCLC, large cells NSCLC, squamous cell carcinoma NSCLC, pleiomorphic cell carcinoma NSCLC and neuroendocrine NSCLC.

In preferred embodiments, a human AMHRII-binding agent is used for treating the above-specified lung cancers that express AMHRII at the cell membrane at a sufficient expression level.

In most preferred embodiments, the said sufficient expression level is expressed as a threshold AMHRII expression score value that is detailed elsewhere in the present specification.

In some embodiments, the said human AMHRII-binding agent consists of an anti-AMHRII monoclonal antibody.

In some embodiments, the said human AMHRII-binding agent consists of an Antibody Drug Conjugate (ADC).

In some embodiments, the said human AMHRII-binding agent consists of an AMHRII-binding engineered receptor.

In some embodiments, the said human AMHRII-binding agent consists of a cell expressing an AMHRII-binding engineered receptor, such as a CAR T-cell or a NK T-cell expressing an AMHRII-binding engineered receptor.

In some embodiments, the said AMHRII-binding agent is combined with one or more distinct anti-cancer agent(s).

This invention also pertains to a method for determining whether an individual is eligible to a lung cancer treatment with an AMHRII-binding agent as defined above, wherein the said method comprises the step of determining whether a lung tumor tissue sample previously obtained from the said individual express the AMHRII protein at the cell surface.

This invention concerns a method for determining whether an individual is responsive to a lung cancer treatment with an AMHRII-binding agent as defined above, wherein the said method comprises the step of determining whether a lung tumor tissue sample previously obtained from the said individual express the AMHRII protein at the cell surface.

### DESCRIPTION OF THE FIGURES

FIG. 1 illustrates the amino acid sequences of the VH and VL domains of a plurality of variants of the 3C23 monoclonal antibody. FIG. 1A illustrates the VH domain of each antibody variant. FIG. 1B illustrates the VL domain of each antibody variant.

FIG. 2 illustrates AMHRII expression by various cancer cell lines.

FIG. 2A illustrates the AMHRII mRNA expression by cancer cell lines. Abscissa: from the left to the right of FIG. 2A: HCT116 (colon colorectal carcinoma), COV434-WT (human ovarian granulosa tumor), K562 (human myelogenous leukemia) and OV90 (human malignant papillary serous adenocarcinoma). Ordinate: AMHRII mRNA expression level as assayed by RT-qPCR, expressed in Arbitrary Units (RQ).

FIGS. 2B to 2F: AMHRII protein membrane expression by the same cancer cell lines as in FIG. 2A: HCT116 (FIG. 2B), COV434-WT (FIG. 2C), K562 (FIG. 2D), NCI-H295R (FIG. 2E) and OV90 (FIG. 2F). Abscissa: fluorescence signal intensity (FL2-A dye) as expressed in Arbitrary Units. Ordinate: cell count.

FIG. 3 illustrates AMHRII expression by various lung cancer cells as measured by flow cytometry.

FIG. 3A illustrates AMHRII protein membrane expression by cells from a squamous cell lung carcinoma patient derived xenograft (Ref Lu7860). FIG. 3B illustrates AMHRII protein membrane expression by cells from a large cell lung carcinoma patient derived xenograft (Ref Lu7166). FIG. 3C illustrates AMHRII protein membrane expression by cells from a squamous cell lung carcinoma patient derived xenograft (Ref Lu7298). FIG. 3D illustrates AMHRII protein membrane expression by cells from a squamous cell lung carcinoma patient derived xenograft (Ref Lu7414). FIG. 3E illustrates AMHRII protein membrane expression by cells from a pleiomorphic cell lung carcinoma patient derived xenograft (Ref Lu7558). Abscissa: fluorescence intensity (FL2-A dye) expressed in Arbitrary Units. Ordinate: cell count.

FIG. 3F illustrates AMHRII protein membrane expression by cells from the healthy margin of the surgically resected human NSCLC (which FACS profile is illustrated in FIG. 3G)

FIG. 3G illustrates AMHRII protein membrane expression by cells from a fresh sample of human NSCLC resected surgically

In FIG. 3: (i) Peak on the left side: cells incubated with an unrelated isotype antibody; (ii) peak on the right sides: cells incubated with the 3C23K anti-AMHRII antibody.

Abscissa: fluorescence intensity (FL2-A dye) expressed in Arbitrary Units. Ordinate: cell count.

FIG. 4 illustrates the relative body weight chances of animals xenografted with human lung cancer cells. Treatments started on day 18 post SC131 implantation. Vehicle and GM102 at 20 mg/kg were biweekly administered i.v. for 3 weeks. Docetaxel at 20 mg/kg was administered slow i.v. once on D0. Cisplatin at 5 mg/kg and gemcitabine at 100 mg/kg were weekly administered i.p. for 1 to 3 weeks. Initial group size: 9 animals. Ordinate: relative body weight as expressed in kg (mean+/−sem). Abscissa: ● Vehicle; ▪ GM102 20 mg/kg; ▴ Docetaxel 20 mg/kg; ▾ Combination of GM102 and Docitaxel; ♦ Combination of Cisplatin 5 mg/kg and Gemcitabine 100 mg/kg; ◯ Combination of GM102, Cisplatin and Gemcitabine.

FIG. 5 illustrates the tumor growth changes induced by the 3C23K anti-AMHRII antibody, in combination or nor with other anti-cancer agents, on animals xenografted with human lung cancer cells. Treatments started on day 18 post SC131 implantation. Vehicle and GM102 at 20 mg/kg were biweekly administered i.v. for 3 weeks. Docetaxel at 20 mg/kg was administered slow i.v. once on D0. Cisplatin at 5 mg/kg and gemcitabine at 100 mg/kg were weekly administered i.p. for 1 to 3 weeks. Initial group size: 9 animals. Ordinate: Tumor volume as expressed in mm3 (mean+/−sem). Abscissa: ● Vehicle; ▪ GM102 20 mg/kg; ▴ Docetaxel 20 mg/kg; ▾ Combination of GM102 and Docitaxel; ♦ Combination of Cisplatin 5 mg/kg and Gemcitabine 100 mg/kg; ◯ Combination of GM102, Cisplatin and Gemcitabine.

FIG. 6 illustrates the anti-tumor activity of the 3C23K anti-AMHRII antibody, in combination or nor with other anti-cancer agents, on animals xenografted with human lung cancer cells. Treatments started on day 18 post SC131 implantation. Vehicle and GM102 at 20 mg/kg were biweekly administered i.v. for 3 weeks. Docetaxel at 20 mg/kg was administered slow i.v. once on D0. Cisplatin at 5 mg/kg and gemcitabine at 100 mg/kg were weekly administered i.p. for 1 to 3 weeks. Initial group size: 9 animals. Ordinate: TC as expressed in percentage. Abscissa: ● Vehicle; ▪ GM102 20 mg/kg; ▴ Docetaxel 20 mg/kg; ▾ Combination of GM102 and Docitaxel; ♦ Combination of Cisplatin 5 mg/kg and Gemcitabine 100 mg/kg; 0 Combination of GM102, Cisplatin and Gemcitabine.

FIG. 7 illustrates the tumor growth changes induced by GM102 (low fucose anti-AMHRII antibody) on animals xenografted with a squamous non-small cell lung cancer tumor xenograft. Each dashed curve: xenografted animals administered with vehicle solution. Each continuous curve: xenografted animals administered with GM102. Abscissa: time period after the beginning of the treatment, as expressed in days. Ordinate: tumor volume, as expressed in mm³.

FIG. 8 illustrates the tumor growth changes induced by GM102 (low fucose anti-AMHRII antibody) on animals xenografted with a squamous non-small cell lung cancer tumor xenograft, at Day 28 after the beginning of the treatment. FIGS. 8A and 8B, ordinate: tumor volume, as expressed in mm³. FIGS. 8A and 8B, abscissa: (i) left side: xenografted animals administered with vehicle solution; (ii) right side: xenografted animals administered with GM102. FIG. 8A: absolute results for each xenografted animal tested. FIG. 8B: mean value+/−Standard Deviation calculated from the results depicted in FIG. 8A.

### DETAILED DESCRIPTION OF THE INVENTION

The inventors have unexpectedly shown that the AMHRII receptor is expressed at the cell membrane of non-small cell lung cancer tissues and especially the epidemoïd NSCLC, adenocarcinoma NSCLC, large cells NSCLC, pleiomorphic cell carcinoma NSCLC, squamous cell carcinoma NSCLC and neuroendocrine NSCLC sub-types. On the opposite, AMHRII was not detected at the membrane level in SCLC or NSCLC from neuroendocrine or acinar subtypes.

The term “AMHR-II” denotes the human Anti-Müllerian Hormone type II Receptor. The sequence of the human AMHR-II is described as SEQ ID NO. 18 herein (lacking the signal peptide MLGSLGLWALLPTAVEA (SEQ ID NO: 17)

As used herein, the term “PDX” is an acronym for the expression “Patient-Derived Xenograft”. Patient-Derived Xenografts are highly used in vivo models of cancers where tissue or cells from a patient's tumor are implanted, i.e. “grafted”, into an immuno-deficient non-human mammal, e.g. an immuno-deficient mouse.

As it is shown in the examples herein, the inventors have found that AMHRII is expressed at the cell membrane of lung cancer tissues with a variable frequency depending of the lung cancer sub-type which is considered.

According to the inventors' knowledge, the membrane expression of AMHRII in lung cancer cells has been shown for the first time herein.

Illustratively, as shown in the examples herein, AMHRII is expressed more frequently by cancer cells derived from tumor tissue originating from patients affected with an epidermoid or an adenocarcinoma NSCLC large cells NSCLC lung cancer than by cancer cells derived from tumor tissue originating from patients affected with a squamous or large cells NSCLC. The relative high frequency detected means that cancer patients affected with one of these four types of lung cancers are more frequently eligible for, i.e., will be more frequently responsive to, an anti-cancer treatment targeting AMHRII, but that such an anti-cancer treatment will be less frequently relevant for treating patients affected with a neuroendocrine NSCLC.

As it is shown in the examples herein, any NSCLC lung cancer may be treated by an AMHRII-binding agent, provided that tumor cells from the said non-gynecologic tumor express AMHRII at their membrane, thus provided that the presence of AMHRII proteins at the tumor cell membrane can be detected or determined according to any method.

Thus, the experimental data provided in the examples herein show that the same AMHRII-binding agent, here an anti-AMHRII monoclonal antibody, is effective for treating a plurality of distinct NSCLC lung cancers provided that the AMHRII target protein is express at the tumor cells membrane.

Incidentally, in the field of anti-cancer active ingredients consisting of target-binding molecules, e.g. target-binding antibodies, the situation wherein the same active ingredient is effective for treating a plurality of distinct cancers is not unprecedented. Illustratively, the anti-PD1 antibody named pembrolizumab has been authorized by the US Food and Drug Administration (FDA) as an active ingredient useful in the treatment of a variety of distinct kinds of cancers, provided that the said cancers share the same physiological features.

As used herein, expression of AMHRII at the cell membrane of lung cancer cells means that the said lung cancer cells express AMHRII at a given quantifiable level or higher than the said quantifiable level.

According to some embodiments, responsiveness of an individual affected with a lung cancer to a treatment with a AMHRII-binding molecule may be assessed by determining whether lung cancer cells from a sample previously collected from the said individual express AMHRII at their membrane.

According to some embodiments, responsiveness of an individual affected with a lung cancer to a treatment with an AMHRII-binding molecule may be assessed by determining whether lung cancer cells from a sample previously collected from the said individual express AMHRII at their membrane above a determined threshold value.

The AMHRII membrane expression level that may be used in some embodiments for determining the responsiveness of a patient affected with a non-gynecologic cancer to a treatment with a AMHRII-binding agent, e.g. an anti-AMHRII antibody, may be assessed with a variety of techniques, which include (i) the percentage of tumor cells contained in a tumor sample that express AMHRII at their membrane, (ii) the mean number of AMHRII proteins at the tumor cell membrane and (iii) the FACS AMHRII signal profile of the tumor cells contained in a tested tumor cell sample.

According to some embodiments, lung cancer cells comprised in a tumor sample previously collected for an individual affected with a lung cancer may be assessed as expressing membranous AMHRII when membranous AMHRII is detected in 5% or more of the lung tumor cells comprised in the said tumor sample.

Thus, in some embodiments, an individual affected with a lung cancer is determined as being responsive to a treatment with an AMHRII-binding agent when 5% or more of the lung tumor cells comprised in a tumor sample previously collected from the said individual express AMHRII at their membrane.

Methods for determining the frequency (e.g. the percentage) of tumor cells expressing membrane AMHRII proteins are disclosed elsewhere in the present specification, including in the examples herein.

According to some embodiments, responsiveness of a patient affected with a lung cancer to a cancer treatment with a AMHRII-binding agent, e.g. an anti-AMHRII antibody, may be assessed by determining the mean number of AMHRII proteins present at the membrane of the tumor cells contained in a tumor sample previously collected from the said patient.

In some embodiments, a patient affected with a lung cancer may be classified as responsive to a treatment with a AMHRII-binding agent, e.g. responsive to a treatment with an anti-AMHRII antibody, when the mean number of membrane AMHRII proteins expressed by the tumor cells contained in a tumor sample previously collected from the said patient is of 10 000 AMHRII proteins or more.

Assessing the number of AMHRII proteins expressed at the lung tumor cell membrane may be performed by using conventional methods comprising (a) a step of incubating a sample containing the cells from a tumor tissue sample previously collected from the patient with a detectable compound that binds specifically with AMHRII protein, such as a fluorescently labeled anti-AMHRII antibody, and further (b) a step of determining the number of the said detectable compounds, e.g. the number of fluorescently labeled anti-AMHRII antibodies, bound to each tested cell from the said sample. Assessing the number of AMHRII proteins expressed at the tumor cell membrane may be, for instance, performed by using the well-known Fluorescence Activated Cell Sorting (FACS) technique, as it is shown in the examples herein.

In still other embodiments, a patient affected with a lung cancer may be classified as responsive to a treatment with a AMHRII-binding agent, e.g. classified as responsive to a treatment with an anti-AMHRII antibody, by analysis of the AMHRII FACS profile of the tumor cells contained in a tumor sample previously collected from the said patient.

According to these still other embodiments, a patient affected with a lung cancer may be classified as responsive to a treatment with a AMHRII-binding agent, e.g. classified as responsive to a treatment with an anti-AMHRII antibody when, in a method of fluorescence activated cell sorting (FACS), the ratio of (i) the mean fluorescence intensity (MFI) value obtained from tumor cells incubated with an isotypic fluorescently labeled antibody to (ii) the mean fluorescence intensity of the tumor cells incubated with an anti-AMHRII fluorescently labeled antibody is of 1.5 or more.

For determining the said mean fluorescence intensity ratio, both the isotypic antibody and the anti-AMHRII antibody are labeled with the same fluorescent agent, such as the Alexa Fluor 488 dye commercialized by the Company ThermoFisher Scientific, as shown in the examples herein.

In some further embodiments, responsiveness of a lung cancer individual to a treatment with an AMHRII-binding agent may be determined by calculating an AMHRII expression score allowing to discriminate between (i) membrane AMHRII-expressing lung cancer cells derived from lung cancers that may be treated with an AMHRII-binding agent and (ii) membrane AMHRII-expressing lung cancer cells derived from lung cancers that may not be treated with an AMHRII-binding agent.

Thus, the inventors have determined that patients affected with a lung cancer who are especially eligible to a cancer treatment with a AMHRII-binding agent described herein, i.e. who are especially responsive to a cancer treatment with a AMHRII-binding agent described herein, encompass those having cancer tumors expressing AMHRII at the cell membrane at a sufficiently high level for consisting in relevant cell targets to be destroyed.

Then, according to these further embodiments, the inventors have determined that a minimal AMHRII expression level measured in a cancer cell sample from a lung cancer patient may confirm that the said patient is responsive to a treatment with an AMHRII-binding agent and that said the patient may thus be treated by an AMHRII-binding agent described herein.

Responsiveness of an individual affected with a lung cancer to a treatment with an AMHRII-binding agent may thus also be determined when AMHRII expression level by lung cancer cells comprised in a sample previously collected from the said individual is assessed by both determining (i) the frequency of tumor cells expressing membranous AMHRII, e.g. the percentage of tumor cells expressing AMHRII at their membrane and (ii) the level of AMHRII membrane expression by the said tumor cells, e.g. the mean number of membranous AMHRII proteins per cell.

Thus, in some of these further embodiments, the inventors have determined that responsiveness of a lung cancer patient to a human AMHRII-binding agent, e.g. to an anti-human AMHRII antibody, requires that, in a sample of tumor cells previously collected from the said patient, (i) the tumor cells contained in the said sample exhibit a minimal mean number of human AMHRII proteins at their membrane and (ii) the frequency of the cells expressing human AMHRII at their membrane, e.g. the percentage of cells expressing human AMHRII at their membrane, if of at least a threshold value.

Accordingly, it is also described herein a further method that may also be used for determining a specific AMHRII expression score value allowing to discriminate between (i) lung cancer patients who are not eligible to a cancer treatment with a AMHRII-binding agent, i.e. lung cancer patients who are not responsive to a cancer treatment with a AMHRII-binding agent, and (ii) lung cancer patients that are eligible to a cancer treatment with a AMHRTT-binding agent, i.e. lung cancer patients who are responsive to a cancer treatment with a AMHRII-binding agent, e.g. an anti-human AMHRII antibody.

More precisely, according to embodiments of the above method, patients affected with a lung cancer described herein and who may be treated against lung cancer with an AMHRII-binding agent as described in the present specification are preferably those for which a membranous AMHRII expression score value is of 1.0 or more has been determined.

The membranous AMHRII expression score may be based on the immuno-histochemical evaluation of the AMHRII expression by the lung cancer cells tested and is the mean value of the membranous AMHRII scores determined from a plurality of lung cancer cell samples originating from distinct individuals affected with a lung cancer, and wherein an individual membranous AMHRII score for a given lung cancer cell sample (i) is assigned as being 0 if no AMHRII expression is detectable, (ii) is assigned as being 1 if a significant AMHRII expression is detected and (iii) is assigned as being 2 if a high AMHRII expression is detected and (iv) is assigned as being 3 if an over-expression of AMHRII is detected.

Indeed, there is a relationship between (i) the score assigned to the membranous AMHRII expression level through the above-described immuno-histochemical evaluation and (ii) the mean number of AMHRII proteins expressed per lung cancer cell. It is shown in the examples herein that the membranous AMHRII expression level, allowing assigning an individual membranous AMHRII score, may also be assessed by determining the mean number of membranous AMHRII proteins per cell, starting from a sample of lung tumor cells that has been previously collected from a lung cancer patient.

According to the above embodiments of determining responsiveness of an individual affected with a lung cancer to a treatment with a AMHRII-binding agent, i.e. to a treatment with an anti-AMHRII antibody, a membranous AMHRII expression score is determined, for a given lung cancer cell sample, by taking into account both (i) the frequency of AMHRII-expressing cells in the said lung cancer cell sample and (ii) the level of membranous AMHRII expression by the said AMHRII-expressing cells. Typically, a membranous AMHRII expression score of a given lung cancer cell sample is determined by the following formula (I):

E-SCORE=FREQ×AMHRII_LEVEL, wherein
E-SCORE means the membranous AMHRII expression score value for a given lung cancer cell sample,
FREQ means the frequency of the cells contained in the said lung cancer cell sample for which membrane AMHRII expression is detected, and
AMHRII_LEVEL means the level of membranous expression of AMHRII by the AMHRII-expressing cells contained in the said given lung cancer cell sample.

Illustratively, a E-SCORE of 1.0 is determined for a given lung cancer cell sample wherein (i) 50% of the cells express AMHRII (FREQ value of 0.5) and (ii) the AMHRII expression level (AMHRII_LEVEL) is of 2.

In some embodiments, an AMHRII expression score (or E-SCORE) is determined by immunohistological methods as shown in the examples herein. According to these preferred embodiments, AMHRII membrane expression is assessed by using a detectable antibody specific for AMHRII and by (i) determining the frequency of cells having the said anti-AMHRII antibody bound thereto and (ii) determining the intensity of the signal generated by the said detectable anti-AMHRII antibody after its binding to the membrane-expressed AMHRII.

Although, as it is shown in the examples herein, AMHRII-expressing lung cancer cells having a membranous AMHRII expression score of 1.0 or more have been determined for various lung cancers, albeit to distinct frequencies

For determining the level of AMHRII membrane expression, detection of AMHRII at the cell membrane shall be most preferably performed by using an anti-AMHRII monoclonal antibody having a high affinity and high specificity for AMHRII, which is illustrated in the examples by the 3C23K anti-AMHRII monoclonal antibody.

Further, determination of AMHRII expression by an immuno-histochemical method with the view of determining a AMHRII score most preferably involves a careful pretreatment of the lung tissue sample before contacting the said sample with an appropriate detection reagent (e.g. a high affinity anti-AMHRII monoclonal antibody such as monoclonal 3C23K antibody having a Kd value of 55.3 pM for binding to AMHRII). Sample pretreatment shall allow increasing the availability to the detection reagent of the AMHRII molecules expressed at the cell surface. Illustratively, as shown in the examples herein, pretreatment method my comprises an appropriate combination of specific steps such as (i) a high-temperature dewaxing by exposure to a microwave source and (ii) a system for amplifying the signal generated by the binding of an AMHRII-binding reagent, such as a biotinylated anti-AMHRII antibody that may be subsequently complexed with a streptavidin-conjugated detectable reagent. A pretreatment dewaxing step has appeared to be important for reversing the detection signal extinction effect due to the prior tissue fixation step. The inventors have shown that AMHRII detectability is particularly sensitive to the action of formalin which is used for the tissue fixation step.

This means that, although a AMHRII-binding agent may be a relevant therapeutic agent for treating patients affected with a lung cancer, it will be preferred to test previously for the AMHRII expression of the tumor-derived lung cancer cells for deciding that a specific patient will be administered with a AMHRII binding agent as described herein.

Further, the inventors have shown that anti-AMHRII antibodies may be advantageously used for treating lung cancers.

Thus, the inventors have shown herein that pharmaceutical agents targeting AMHRII are useful as novel therapeutic tools for preventing or treating these kind of cancers, and especially a NSCLC selected in a group comprising epidermoid NSCLC, pleiomorphic cell carcinoma NSCLC and neuroendocrine NSCLC adenocarcinoma NSCLC, large cells NSCLC and squamous cell carcinoma NSCLC and neuroendocrine NSCLC.

According to the invention, the expression “comprising”, such as in “comprising the steps of”, is also understood as “consisting of”, such as in “consisting of the steps of” is also understood as “consisting of”, such as “consisting of the steps of”.

The AMH receptor (AMHR or AMHR2 or AMHRII) is a serine/threonine kinase with a single transmembrane domain belonging to the family of type II receptors for TGF-beta-related proteins. Type II receptors bind the ligand on their own but require the presence of a type I receptor for signal transduction. Imbeaud et al. (1995, Nature Genet, Vol. 11: 382-388,) cloned the human AMH type II receptor gene. The human AMH receptor protein consists of 573 amino acids: 17, 127, 26, and 403 of the 573 amino acids form a signal sequence, extracellular domain (ECD), transmembrane domain, and intracellular domain containing a serine/threonine kinase domain, respectively

As used herein, the term “AMHRII” refers to the human Anti-Müllerian Hormone Type II Receptor having the amino acid sequence of SEQ ID NO. 17.

Expression of anti-Müllerian hormone receptor (AMHRII) was already described in the art in gynecologic cancers, tumors which are largely infiltrated by immune myeloid cells. AMHRII has been identified as a target molecule for treating gynecologic cancers. Antibodies directed to AMHRII have been produced as therapeutic tools for treating these cancers. It may be cited notably the 12G4 anti-AMHRII antibody and variants thereof described in the PCT applications n^{o} WO 2008/053330 and n^{o} WO 2011/141653 for treating ovarian cancers, as well as the 3C23K anti-AMHRII antibody described in the PCT application. It may also be mentioned the PCT application n^{o} WO 2017/025458 which disclosed a specific treatment strategy against ovarian cancer by using anti-AMHRII antibody drug conjugates.

Expression of anti-Müllerian hormone receptor gene (AMHRII gene) was also described by Beck et al. (2016, Cell Reports, Vol. 16: 657-671). These authors have shown that AMH signaling was an important contributor to epithelial plasticity, survival signaling, and selective drug resistance in NSCLC. The work of Beck et al. (2016) offered insights into intracellular mechanisms of NSCLC pathogenesis, notably by reporting, through modulating the expression of various genes of interest by using siRNAs, the identification and characterization of a previously undefined autocrine signaling axis in a subset of NSCLC tumors, involving anti-Müllerian hormone, and its type II receptor, as important for response to the Hsp90 inhibitor ganetespib and to the approved chemotherapeutic cisplatin. These authors also found, through Western blotting experiments, a low abundance of AMH and AMHR2 proteins present within the cells of three cell lines, namely A549 and H1299, which production is blocked by targeting the respective genes by SiRNAs.

The inventors have now unexpectedly found that AMHRII was also expressed at the surface of various human lung cancer cells, which include especially non-small cell lung cancer (NSCLC) cells and even more especially a NSCLC selected in a group comprising epidermoid NSCLC, pleiomorphic cell carcinoma NSCLC and neuroendocrine NSCLC adenocarcinoma NSCLC, large cells NSCLC and squamous cell carcinoma NSCLC and neuroendocrine NSCLC. The inventors have also shown that there is no relationship between (i) the AMHRII gene expression by cancer cells and (ii) the cell membrane AMHRII protein expression by the same cancer cells.

The inventors' findings regarding AMHRII surface expression by human lung cancer cells notably derive from immunohistochemical assays with an anti-AMHRII antibody that were performed by using human lung tumor tissue samples previously obtained from lung cancer patients. The inventors' findings relating to AMHRII surface expression by human lung cancer cells were also obtained from immunohistochemical assays with an anti-AMHRII antibody that were performed on lung tumor tissue samples originating from human primary lung cancer cells xenografts in mice.

The present inventors have also shown that anti-AMHRII antibodies are useful for treating human lung cancers that express AMHRII at the tumor cell surface, and especially those AMHRII-expressing lung cancers disclosed in the present specification, which include non-small cell lung cancer and especially epidermoid NSCLC, pleiomorphic cell carcinoma NSCLC and neuroendocrine NSCLC adenocarcinoma NSCLC, large cells NSCLC and squamous cell carcinoma NSCLC and neuroendocrine NSCLC. Notably, good anti-cancer activity has been shown with anti-AMHRII antibodies, as well with anti-AMHRII antibodies combined with a chemical anti-cancer agent such as the well-known anti-cancer agents docetaxel, cisplatin and/or gemcitabine.

The inventors have shown that an anti-AMHRII antibody that had proved anti-tumor efficacy against AMHRII-expressing gynecologic cancers in the art is also useful for preventing or treating AMHRII-expressing lung cancers, and especially those AMHRII-expressing lung cancers disclosed in the present specification, such as non-small cell lung cancer and especially epidermoid NSCLC, adenocarcinoma NSCLC, large cells NSCLC and squamous cell carcinoma NSCLC, pleiomorphic cell carcinoma NSCLC and neuroendocrine NSCLC.

More precisely, it is shown in the examples herein that the anti-AMHRII antibody named 3C23K exerts an anti-tumor activity in vivo against human lung cancer, and especially against non-small cell lung cancers disclosed herein, including when the said anti-AMHRII antibody treatment is combined with a treatment with one or more distinct anti-cancer agent(s) such as docetaxel, cisplatine and/or gemcitabine.

Still further, the inventors have also shown that the anti-AMHRII 3C23K antibody induces no detectable toxic event in vivo, and notably no significant body weight loss.

Thus, the present invention relates to a human AMHRII-binding agent for its use for preventing or treating a lung cancer, especially a non-small cell lung cancer (NSCLC) and more specifically non-small cell lung cancers (NSCLC) selected in a group comprising epidermoid NSCLC, adenocarcinoma NSCLC, large cells NSCLC, squamous cell carcinoma NSCLC, pleiomorphic cell carcinoma and neuroendocrine NSCLC.

This invention also concerns the use of a human AMHRII-binding agent for the preparation of a medicament for preventing or treating a lung cancer, and especially a lung cancer selected in a group comprising epidermoid NSCLC, adenocarcinoma NSCLC, large cells NSCLC, squamous cell carcinoma NSCLC, pleiomorphic cell carcinoma NSCLC and neuroendocrine NSCLC.

This invention also pertains to a method for preventing or treating lung cancer, and especially a lung cancer selected in a group comprising epidermoid NSCLC, adenocarcinoma NSCLC, large cells NSCLC, squamous cell carcinoma NSCLC, pleiomorphic cell carcinoma NSCLC and neuroendocrine NSCLC, wherein the said method comprises a step of administering to an individual in need thereof an AMHRII-biding agent as disclosed in the present specification.

An AMHRII-binding agent that may be used according to the present invention does not require a mimicking of the MIS natural ligand activity. Thus, there is no need that an AMHRII-binding agent that may be used according to the invention activates any cell signaling pathway upon its binding to AMHRII. Instead, sole the ability of the said agent to bind to AMHRII is required, since the said agent is used exclusively for targeting a cytotoxicity-inducing activity, such as a cytotoxicity-inducing entity, which encompasses an anti-AMHRII cytotoxic immuno-conjugate, an ADCC-inducing or an ADC-inducing anti-AMHRII antibody or a CAR T-cell expressing an AMHRII-binding engineered receptor.

AMHRII Binding Agent

As used herein, an AMHRII-binding agent encompasses any agent that specifically binds to AMHRII and which, when presented in an appropriate manner, will cause the death of the target cells expressing AMHRII at their surface after that the said agent has bound the cell membrane-expressed AMHRII.

An AMHRII-binding agent that is used for treating a lung cancer as described herein may also be termed a “therapeutic AMHRII-binding agent” herein.

Generally, a AMHRII-binding agent encompasses a protein or a nucleic acid that specifically binds to AMHRII.

AMHRII-binding proteins mainly encompass protein comprising one or more Complementary Determining Regions (CDRs) that originate from an anti-AMHRII antibody or from an AMHRII-binding fragment of an anti-AMHRII antibody, it being understood that the said AMHRII-binding proteins may be expressed as Chimeric Antigen Receptors (CARS) by engineered cells such as CAR-T-cells, NK T-cells or CAR Macrophages.

AMHRII-binding nucleic acids mainly encompass nucleic acid aptamers that have been especially selected for their specific binding properties to AMHRII.

In some preferred embodiments, the AMHRII-binding agent is an anti-AMHRII antibody or an AMHRII-binding fragment thereof.

In most preferred embodiments, the AMHRII-binding agent is an anti-AMHRII monoclonal antibody or an AMHRII-binding fragment thereof.

According to these preferred embodiments, anti-AMHRII monoclonal antibodies encompass chimeric anti-AMHRII antibodies, humanized anti-AMHRII antibodies and human AMHRII antibodies, as well as the AMHRII-binding fragments and AMHRII-binding derivatives thereof.

Various AMHRII antibodies are known in the art and may be used according to the invention as AMHRII-binding agents. For the purpose of performing the present invention, the one skilled in the art may use, for illustration, the recombinant human anti-AMHRII marketed by Creative Biolabs under the reference n^{o} MHH-57.

In some embodiments, an anti-AMHRII antibody that may be used according to the invention is the humanized 12G4 antibody disclosed in the PCT application n^{o} WO 2008/053330.

In some other embodiments, the said anti-AMHRII antibodies are the humanized antibodies described in the PCT application n^{o} WO 2011/141653, which humanized antibodies encompass the 3C23 antibodies as well as the variants thereof, which variants thereof include the 3C23K humanized antibody.

In still further embodiments, the said anti-AMHRII antibodies are those described in the PCT application n^{o} WO 2017/025458. According to these further embodiments, the PCT application n^{o} WO 2017/025458 disclosed AMHRII-binding agents under the form of Antibody Druc Conjugates (ADC) wherein the said anti-AMHRII antibodies are linked to a cytotoxic agent.

A monoclonal antibody against Mullerian Hormone type II receptor (and humanized derivatives thereof) has been developed in the art for the treatment of ovarian cancer (see EP 2097453B1 and U.S. Pat. No. 8,278,423, which is hereby incorporated by reference in its entirety).

Among the AMHRII-binding agents that may be used according to the invention, the one skilled in the art may use the monoclonal antibody 12G4 (mAb 12G4), or chimeric or humanized variants thereof, including such an antibody which has been derivatized with a drug or detectable label to form an ADC. The hybridoma producing mAb12G4 has been deposited at the Collection Nationale de Cultures de Microorganismes (CNCM, Institut Pasteur, 25 rue du Docteur Roux, 75724 Paris Cedex 15, France), in accordance with the terms of Budapest Treaty, on the 26 Sep. 2006) and has CNCM deposit number 1-3673. The variable domain of the light and heavy chains of the mAb 12G4 have been sequenced as have been the complementarity determining regions (CDRs) of mAb 12G4 (see EP 2097453B1 and U.S. Pat. No. 8,278,423, which is hereby incorporated by reference in its entirety). mAb 12G4 and its chimeric or humanized variants can be used for the production of ADC as disclosed herein.

The PCT application n^{o} PCT/FR2011/050745 (International Publication n° WO/2011/141653) and U.S. Pat. No. 9,012,607, each of which is hereby incorporated by reference in its entirety, disclose novel humanized antibodies that are derived from the murine 12G4 antibody. These humanized antibodies may be used as AMHRII-binding agents for the purpose of the present invention. In particular embodiments disclosed in the PCT application n^{o} WO/2011/141653, the antibodies are those identified as the 3C23 and 3C23K. The nucleic acid sequences and polypeptide sequences of these antibodies are provided as SEQ ID NOs: 1-16 herein. In some aspects of the invention, the anti-AMHRII antibodies of interest may be referred to as “comprising a light chain comprising SEQ ID NO: and a heavy chain comprising SEQ ID NO:”. Thus, in various embodiments, particularly preferred antibodies, including for the generation of ADC, comprise:
a) a light chain comprising SEQ ID NO: 2 and a heavy chain comprising SEQ ID NO: 4 (3C23 VL and VH sequences without leaders);
b) a light chain comprising SEQ ID NO: 6 and a heavy chain comprising SEQ ID NO: 8 (3C23K VL and VH sequences without leaders);
c) a light chain comprising SEQ ID NO: 10 and a heavy chain comprising SEQ ID NO: 12 (3C23 light and heavy chains without leaders);
d) a light chain comprising SEQ ID NO: 14 and a heavy chain comprising SEQ ID NO: 16 (3C23K light and heavy chains without leaders).

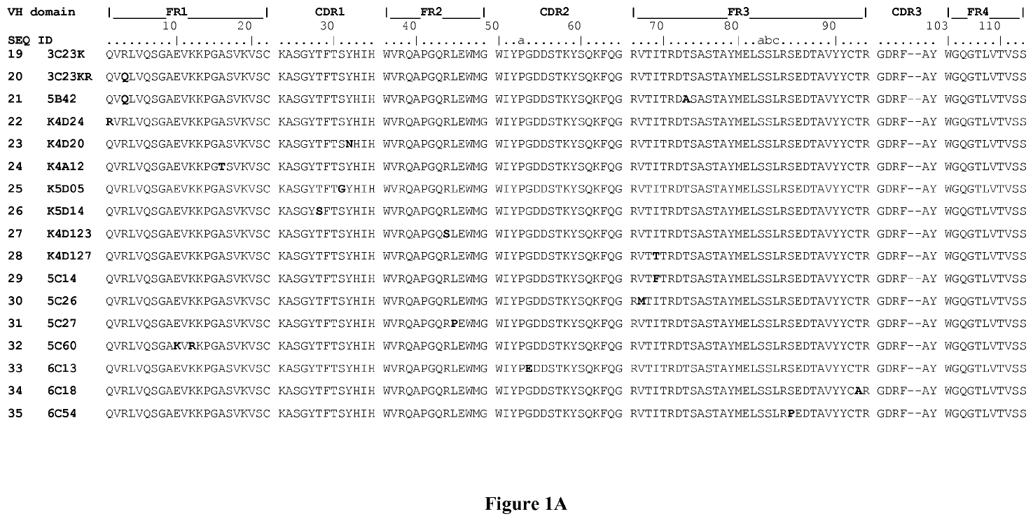

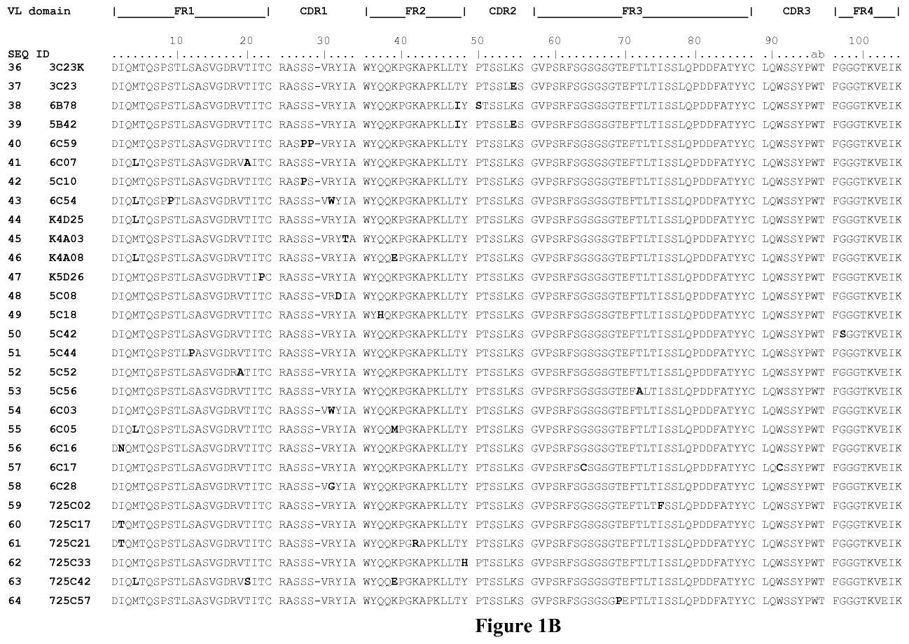

Other antibodies (e.g., humanized or chimeric antibodies) can be based upon the heavy and light chain sequences provided in FIGS. 1A and 1B (e.g., antibodies, such as humanized or chimeric antibodies containing the CDR sequences disclosed within the Figures) can be used as anti-MAHRII-binding agents of interest, including for the formation of ADCs. Thus, the invention also pertains to the use of anti-AMHRII antibodies comprising/containing CDRs comprising (or consisting of) the following sequences:

-CDRL-1:
(SEQ ID NO. 65)
RASX1X2VX3X4X5A, where X1 and X2 are,
independently, S or P, X3is R or W or G, X4is T or
D, and X5is I or T;
-CDRL-2 is
(SEQ ID NO. 66)
PTSSLX6S where X6 is K or E;
and
-CDRL-3 is
(SEQ ID NO. 67)
LQWSSYPWT;
-CDRH-1 is
(SEQ ID NO. 68)
KASGYX7FTX8X9HIH where X7 is S or T, X8 is S or G
and X9 is Y or N;
-CDRH-2 is
(SEQ ID NO. 69)
WIYPX10DDSTKYSQKFQG where X10 is G or E
and
-CDRH-3 is
(SEQ ID NO. 70)
GDRFAY

This invention also relates to the use of ADCs generated using such anti-AMHRII antibodies for treating lung cancer, and especially non-small cell lung cancer and small cell lung cancer. Antibodies (e.g., chimeric or humanized) within the scope of this application include those disclosed in the following table: Alternatively, human monoclonal antibodies that specifically bind to AMHR-II can be used for the preparation of ADCs. 3C23K antibody is defined by:
SEQ ID NO: 19 for VH amino acid sequence
SEQ ID NO: 36 for VL amino acid sequence

Table 1 hereunder lists anti-AMHRII humanized antibodies that may be used according to the invention.

TABLE 1
anti-AMHRII antibodies

| | Mutations | | | |
|---|---|---|---|---|
| | | SEQ ID in | | SEQ ID in |
| | | sequence | | sequence |
| Antibody | VH mutations | listing | VL mutations | listing |
| 3C23K | | 19 | | 36 |
| 3C23 | | 19 | L-K55E | 37 |
| 3C23KR | H-R3Q | 20 | | 36 |
| 6B78 | H-R3Q | 20 | L-T48I, L-P50S | 38 |
| 5B42 | H-R3Q, | 21 | L-T48I, L-K55E | 39 |
| | H-T73A | | | |
| K4D-24 | H-Q1R | 22 | | 36 |
| 6C59 | H-Q1R | 22 | L-S27P, L-S28P | 40 |
| K4D-20 | H-Y32N | 23 | | 36 |
| K4A-12 | H-A16T | 24 | | 36 |
| K5D-05 | H-S31G | 25 | | 36 |
| K5D-14 | H-T28S | 26 | | 36 |
| K4D-123 | H-R44S | 27 | | 36 |
| K4D-127 | H-I69T | 28 | | 36 |
| 6C07 | H-I69T | 28 | L-M4L, L-T20A | 41 |
| 5C14 | H-I69F | 29 | | 36 |
| 5C26 | H-V67M | 30 | L-S27P | 42 |
| 5C27 | H-L45P | 31 | | 36 |
| 5C60 | H-E10K, | 32 | | 36 |
| | H-K12R | | | |
| 6C13 | H-G53E | 33 | | 36 |
| 6C18 | H-T93A | 34 | | 36 |
| 6C54 | H-S84P | 35 | L-M4L, L-S9P, | 43 |
| | | | L-R31W | |
| K4D-25 | | 19 | L-M4L | 44 |
| K4A-03 | | 19 | L-I33T | 45 |
| K4A-08 | | 19 | L-M4L, L-K39E | 46 |
| K5D-26 | | 19 | L-T22P | 47 |
| 5C08 | | 19 | L-Y32D | 48 |
| 5C10 | | 19 | L-S27P | 42 |
| 5C18 | | 19 | L-Q37H | 49 |
| 5C42 | | 19 | L-G97S | 50 |
| 5C44 | | 19 | L-S12P | 51 |
| 5C52 | | 19 | L-19A | 52 |
| 5C56 | | 19 | L-T72A | 53 |
| 6C03 | | 19 | L-R31W | 54 |
| 6C05 | | 19 | L-M4L, L-M39K | 55 |
| 6C16 | | 19 | L-I2N | 56 |
| 6C17 | | 19 | L-G63C, L-W91C | 57 |
| 6C28 | | 19 | L-R31G | 58 |
| 725C02 | | 19 | L-I75F | 59 |
| 725C17 | | 19 | L-I2T | 60 |
| 725C21 | | 19 | L-I2T, L-K42R | 61 |
| 725C33 | | 19 | L-Y49H | 62 |
| 725C42 | | 19 | L-M4L, L-T20S, | 63 |
| | | | L-K39E | |
| 725C44 | | 19 | L-S27P | 42 |
| 725C57 | | 19 | L-T69P | 64 |

Anti-AMHRII Antibodies, AMHRII-Binding Fragments or AMHRII-Binding Derivatives of Anti-AMHRII Antibodies

The term “antibody” is used in the broadest sense and includes monoclonal antibodies (including full length or intact monoclonal antibodies), polyclonal antibodies, multivalent antibodies, multispecific antibodies (e.g., bispecific antibodies), and antibody fragments (see below) so long as they exhibit the desired biological activity.

Thus, as used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to AMHRII to the substantial exclusion of binding to other molecules (i.e. molecules unrelated to AMHRII). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 7^{th} Ed., W.H. Freeman & Co., New York, 2013.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with the invention may be made by the hybridoma method first described by Kohler et al, Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature 352:624-628 (1991) or Marks et al, J. MoI Biol. 222:581-597 (1991), for example.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, κ and λ light chains refer to the two major antibody light chain isotypes.

As used herein the term “complementarity determining region” or “CDR” refers to the part of the two variable chains of antibodies (heavy and light chains) that recognize and bind to the particular antigen. The CDRs are the most variable portion of the variable chains and provide the antibody with its specificity. There are three CDRs on each of the variable heavy (VH) and variable light (VL) chains and thus there are a total of six CDRs per antibody molecule. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VHCDR3 is located in the variable domain of the heavy chain of the antibody in which it is found, whereas a VLCDR1 is the CDR1 from the variable domain of the light chain of the antibody in which it is found. An antibody that binds LHR will have a specific VH region and the VL region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues.

“Single-chain Fv” or “scFv” antibody fragments comprise the VH and VL domains of antibody, wherein these domains are present in a single polypeptide chain. Generally the Fv polypeptide further comprises a polypeptide linker between the VH and VL domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (VH) connected to a light chain variable domain (VL) in the same polypeptide chain (VH and VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al, Proc. Natl. Acad. Sci. USA, 90:6444-6448 (1993).

Diabodies or bi-specific antibodies can be roughly divided into two categories: immunoglobulin G (IgG)-like molecules and non-IgG-like molecules. IgG-like bsAbs retain Fc-mediated effector functions such as antibody-dependent cell-mediated cytotoxicity (ADCC), complement-dependent cytotoxicity (CDC), and antibody-dependent cellular phagocytosis (ADCP) (Spiess et al., 2015, Mol Immunol., Vol. 67(2): 95-106.). The Fc region of bsAbs facilitates purification and improves solubility and stability. Bi-specific antobodies in IgG-like formats usually have longer serum half-lives owing to their larger size and FcRn-mediated recycling (Kontermann et al., 2015, Bispecific antibodies. Drug Discov Today Vol. 20(7): 838-47). Non-IgG-like bsAbs are smaller in size, leading to enhanced tissue penetration (Kontermann et al., 2015, Bispecific antibodies. Drug Discov Today Vol. 20(7): 838-47).

According to some preferred embodiments, bispecific antibodies according to the invention comprise (i) a first antigen binding site that binds to AMHRII and (ii) a second antigen binding site that binds to a target antigen which is distinct from AMHRII and especially a target antigen that may be expressed by cancer cells or immune cells of the tumor microenvironment such as T-cells, NK or macrophages. In some embodiments, in such bispecific antibodies, the said second antigen binding site binds to a target antigen which is CD3 and allows the engagement of T-cells. This target antigen can also be PDL1 to unlock T-cells or CD16 to activate NK or macrophages.

The monoclonal antibodies specified herein specifically include “chimeric” anti-AMHRII antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

The monoclonal antibodies specified herein also encompass humanized anti-AMHRII antibodies. “Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al, Nature 321:522-525 (1986); Riechmann et al., Nature 332:323-329 (1988); and Presta, Curr. Op. Struct. Biol. 2:593-596 (1992).

The monoclonal anti-AMHRII antibodies specified herein further encompass anti-AMHRII human antibodies. A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. MoI. Biol, 227:381 (1991); Marks et al., J. MoI. Biol, 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al, Monoclonal Antibodies and Cancer Therapy, Alan R. Liss, p. 77 (1985); Boerner et al., J. Immunol, 147 (1):86-95 (1991); and U.S. Pat. No. 5,750,373.

As used herein, “antibody mutant” or “antibody variant” refers to an amino acid sequence variant of the species-dependent antibody wherein one or more of the amino acid residues of the species-dependent antibody have been modified. Such mutants necessarily have less than 100% sequence identity or similarity with the species-dependent antibody. In one embodiment, the antibody mutant will have an amino acid sequence having at least 75% amino acid sequence identity or similarity with the amino acid sequence of either the heavy or light chain variable domain of the species-dependent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e same residue) or similar (i.e. amino acid residue from the same group based on common side-chain properties, see below) with the species-dependent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

Humanized antibodies may be produced by obtaining nucleic acid sequences encoding CDR domains and constructing a humanized antibody according to techniques known in the art. Methods for producing humanized antibodies based on conventional recombinant DNA and gene transfection techniques are well known in the art (See, e.g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

It may be desirable to modify an anti-AMHRII antibody specified herein with respect to effector function, e.g. so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al, J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989). WO00/42072 (Presta, L.) describes antibodies with improved ADCC function in the presence of human effector cells, where the antibodies comprise amino acid substitutions in the Fc region thereof. Preferably, the antibody with improved ADCC comprises substitutions at positions 298, 333, and/or 334 of the Fc region (Eu numbering of residues). Preferably the altered Fc region is a human IgG1 Fc region comprising or consisting of substitutions at one, two or three of these positions. Such substitutions are optionally combined with substitution(s) which increase CIq binding and/or CDC.

Antibodies with altered CIq binding and/or complement dependent cytotoxicity (CDC) are described in WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al). The antibodies comprise an amino acid substitution at one or more of amino acid positions 270, 322, 326, 327, 329, 313, 333 and/or 334 of the Fc region thereof (Eu numbering of residues).

In some embodiments, AMHRII-binding agents encompass glyco-engineered anti-AMHRII antibodies.

As used herein, the term “glycoengineering” refers to any art-recognized method for altering the glycoform profile of a binding protein composition. Such methods include expressing a binding protein composition in a genetically engineered host cell (e.g., a CHO cell) that has been genetically engineered to express a heterologous glycosyltransferase or glycosidase. In other embodiments, the glycoengineering methods comprise culturing a host cell under conditions that bias for particular glycoform profiles.

As used herein, a “glyco-engineered antibody” encompasses (i) an antibody comprising a hyper-galactosylated Fc fragment, (ii) an antibody comprising a hypo mannosylated Fc fragment, which encompasses a amannosylated Fc fragment, and (iii) an antibody comprising a hypo fucosylated Fc fragment, which encompasses an afucosylated Fc fragment. As used herein, a glyco-engineered fragment encompasses a Fc fragment having an altered glycosylation which is selected in a group comprising one or more of the following altered glycosylation (i) hyper-galactosylation, (ii) hypo-mannosylation and (iii) hypo-fucosylation. Consequently, a glyco-engineered Fc fragment from an anti-AMHRII antibody as used according to the invention encompass the illustrative examples of a hyper-galactosylated, a hypo-mannosylated and a hypo-fucosylated Fc fragment.

The one skilled in the art may refer to well-known techniques for obtaining anti-AMHRII antibodies comprising hyper-galactosylated Fc fragments, hypo mannosylated Fc fragments and hypo fucosylated Fc fragments that are known to bind to Fc receptors with a higher affinity than non-modified Fc fragments.

Glyco-engineered anti-AMHRII antibodies encompass anti-AMHRII antibodies comprising a hypofucosylated Fc fragment, which may also be termed a “low fucose” Fc fragment.

Immunoconjugates, Especially Antibody Drug Conjugates (ADC)

AMHRII-binding agents that may be used for the purpose of the present invention encompass antibodies specified herein that are conjugated to a cytotoxic agent such as a chemotherapeutic agent, toxin (e.g. an enzymatically active toxin of bacterial, fungal, plant or animal origin, or fragments thereof), or a radioactive isotope (i.e., a radio conjugate). Such antibody conjugates encompass those described in the PCT application n^{o} WO 2017/025458. The PCT application n^{o} WO 2017/025458 notably disclosed the anti-AMHRII 3C23K antibody, as well as 3C23K ADC conjugates, for which in vivo anti-cancer activity is shown herein against non-gynecologic human cancers.

Cytotoxic agents encompass enzymatically active toxins. Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes.

A variety of radionuclides are available for the production of radioconjugate antibodies.

Conjugates of the antibody and cytotoxic agent are made using a variety of bifunctional protein coupling agents such as those disclosed in the PCT application n^{o} WO 2017/025458.

Preferred immunoconjugates of anti-AMHRII ADC antibody conjugates are those described in the PCT application n^{o} WO 2017/025458.

CAR Cells, Including CAR T-Cells, CAR NK Cells and CAR Macrophages

In some embodiments, the human-AMHRII-binding agent is an AMHRII-binding receptor or an AMHRII-binding receptor-expressing cell, and especially an AMHRII-binding receptor-expressing CAR T-cell, an AMHRII-binding receptor NK cell, or an AMHRII-binding receptor-expressing CAR Macrophage.

Thus, in some embodiments, the human AMHRII-binding agent is an AMHRII-binding engineered receptor, and most preferably an AMHRII-binding engineered receptor for which the AMHRII-binding region thereof derives from a monoclonal anti-AMHRII antibody disclosed in the present specification.

Typically, the AMHRII-binding engineered receptor consists of a Chimeric Antigen Receptor (CAR) comprising (i) an extracellular domain, (ii) a transmembrane domain and (iii) an intracellular domain, and wherein the extracellular domain is an AMHRII-binding moiety which derives from an anti-AMHRII monoclonal antibody disclosed in the present specification. In some embodiments, the extracellular domain of the said AMHRII-binding engineered receptor comprises (i) an antibody VH chain comprising the CDRs derived from an anti-AMHRII monoclonal antibody disclosed herein and (ii) an antibody VL chain comprising the CDRs derived from an anti-AMHRII monoclonal antibody disclosed herein. In some embodiments, the extracellular domain of the said AMHRII-binding engineered receptor comprises the VH chain and the VL chain of an anti-AMHRII monoclonal antibody disclosed herein. In some embodiments, the extracellular domain of the said AMHRII-binding engineered receptor is a ScFv comprising the CDRs derived from the VH chain and the CH chain from an anti-AMHRII monoclonal antibody disclosed in the present specification, respectively. In some embodiments, the extracellular domain of the said AMHRII-binding engineered receptor is a ScFv comprising the VH chain and the CH chain from an anti-AMHRII monoclonal antibody disclosed in the present specification, respectively.

Is also encompassed herein an AMHRII-binding agent consisting of a cell expressing such an AMHRII-binding receptor, and especially a CAR T-cell, a CAR NK-cell or a CAR Macrophage expressing such an AMHRII-binding receptor.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to AMHRII” means any oligopeptide or polypeptide that can bind to AMHRII. The “intracellular domain” means any oligopeptide or polypeptide known to function as a domain that transmits a signal to cause activation or inhibition of a biological process in a cell. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains.

CAR T-cells are genetically engineered autologous T-cells in which single chain antibody fragments (scFv) or ligands are attached to the T-cell signaling domain capable of facilitating T-cell activation (Maher, J. (2012) ISRN Oncol. 2012:278093; Curran, K. J. et al. (2012) J. Gene Med. 14:405-415; Fedorov, V. D. et al. (2014) Cancer J. 20:160-165; Barrett, D. M. et al. (2014) Annu. Rev. Med. 65:333-347).

By “intracellular signaling domain” is meant the portion of the CAR that is found or is engineered to be found inside the T cell. The “intracellular signaling domain” may or may not also contain a “transmembrane domain” which anchors the CAR in the plasma membrane of a T cell. In one embodiment, the “transmembrane domain” and the “intracellular signaling domain” are derived from the same protein (e.g. CD3ζ) in other embodiments; the intracellular signaling domain and the transmembrane domain are derived from different proteins (e.g. the transmembrane domain of a CD3ζ and intracellular signaling domain of a CD28 molecule, or vice versa).

By “co-stimulatory endodomain” is meant an intracellular signaling domain or fragment thereof that is derived from a T cell costimulatory molecule. A non-limiting list of T cell costimulatory molecules include CD3, CD28, OX-40, 4-1BB, CD27, CD270, CD30 and ICOS. The co-stimulatory endodomain may or may not include a transmembrane domain from the same or different co-stimulatory endodomain.

By “extracellular antigen binding domain” is meant the portion of the CAR that specifically recognizes and binds to AMHRII.

In preferred embodiments, the “extracellular binding domain” is derived from an anti-AMHRII monoclonal antibody. For example, the “extracellular binding domain” may include all or part of a Fab domain from a monoclonal antibody. In certain embodiments, the “extracellular binding domain” includes the complementarity determining regions of a particular anti-AMHRII monoclonal antibody. In still another embodiment, the “extracellular binding domain” is a single-chain variable fragment (scFv) obtained from an anti-AMHRII monoclonal antibody specified herein.

In preferred embodiments, the extracellular binding domain is derived from any one of the anti-AMHRII monoclonal antibodies described in the present specification and especially from the 3C23K anti-AMHRII monoclonal antibody.

I. Extracellular Antigen Binding Domain

In one embodiment, the CAR of the current invention comprises an extracellular antigen binding domain from one of the anti-AMHRII monoclonal antibodies described herein.

In one embodiment, the extracellular binding domain comprises the following CDR sequences:

-CDRL-1:
(SEQ ID NO. 65)
RASX1X2VX3X4X5A, where X1 and X2are,
independently, S or P, X3is R or W or G, X4is T or
D, and X5is I or T;
-CDRL-2 is
(SEQ ID NO. 66)
PTSSLX6S where X6 is K or E;
and
-CDRL-3 is
(SEQ ID NO. 67)
LQWSSYPWT;
-CDRH-1 is
(SEQ ID NO. 68)
KASGYX7FTX8X9HIH where X7 is S or T, X8 is S or G
and X9 is Y or N;
-CDRH-2 is
(SEQ ID NO. 69)
WIYPX10DDSTKYSQKFQG where X10 is G or E
and
-CDRH-3 is
(SEQ ID NO. 70)
GDRFAY

II. Linker Between VL and VH Domains of KappaMab scFv

In a further embodiment, the anti-AMHRII VL is linked to the anti-AMHRII VH via a flexible linker. Specifically, the flexible linker is a glycine/serine linker of about 10-30 amino acids (for example 30, 25, 20, 19, 18, 17, 16, 15, 14, 13, 12, 11, 10, 9, 8, 7, 6, or 5 amino acids) and comprises the structure (Gly4Ser)³.

III. Spacers Between Extracellular Antigen Binding Domain and Intracellular Signaling Domain

The extracellular antigen binding domain is linked to the intracellular signaling domain by the use of a “spacer”. The spacer is designed to be flexible enough to allow for orientation of the antigen binding domain in such a way as facilitates antigen recognition and binding. The spacer may derive from the anti-AMHRII immunoglobulins themselves and can include the IgG1 hinge region or the CH2 and/or CH3 region of an IgG.

IV. Intracellular Signaling Domain

The intracellular signaling domain comprises all or part of the CD3 chain. CD, also known as CD247, together with either the CD4 or CD8 T cell co-receptor is responsible for coupling extracellular antigen recognition to intracellular signaling cascades.

In addition to the including of the CD3ζ signaling domain, the inclusion of co-stimulatory molecules has been shown to enhance CAR T-cell activity in murine models and clinical trials. Several have been investigated including CD28, 4-IBB, ICOS, CD27, CD270, CD30 and OX-40.

In certain embodiments, methods of producing CAR expressing cells are disclosed comprising, or alternatively consisting essentially of: (i) transducing a population of isolated cells with a nucleic acid sequence encoding a CAR and (ii) selecting a subpopulation of cells that have been successfully transduced with said nucleic acid sequence of step (i). In some embodiments, the isolated cells are T-cells, an animal T-cell, a mammalian T-cell, a feline T-cell, a canine T-cell or a human T-cell, thereby producing CAR T-cells. In certain embodiments, the isolated cell is an NK-cell, e.g., an animal NK-cell, a mammalian NK-cell, a feline NK-cell, a canine NK-cell or a human NK-cell, thereby producing CAR NK-cells.

Therapeutic Applications of CAR T-Cells, CAR NK Tcells and CAR Macrophages.

The CAR cells, which include the CAR T-cells, the CAR NK cells and the CAR Macrophages described herein, may be used to treat AMHRII-expressing lung tumors. The CAR cells of the present invention are preferably used for treating AMHRII-expressing lung tumors in patients affected with a lung cancer described herein, and especially a non-small cell lung cancer or a small cell lung cancer.

The CAR cells of the present invention may be administered either alone or in combination with diluents, known anti-cancer therapeutics, and/or with other components such as cytokines or other cell populations that are immunostimulatory.

Method aspects of the present disclosure relate to methods for inhibiting the growth of a tumor in a subject in need thereof and/or for treating a cancer patient in need thereof. In some embodiments, the tumor is a solid lung tumor.

The CAR cells as disclosed herein may be administered either alone or in combination with diluents, known anti-cancer therapeutics, and/or with other components such as cytokines or other cell populations that are immunostimulatory. They may be first line, second line, third line, fourth line, or further therapy. The can be combined with other therapies. Non-limiting examples of such include chemotherapies or biologics. Appropriate treatment regimen will be determined by the treating physician or veterinarian.

Pharmaceutical compositions comprising the CAR of the present invention may be administered in a manner appropriate to the disease to be treated or prevented. The quantity and frequency of administration will be determined by such factors as the condition of the patient, and the type and severity of the patient's disease, although appropriate dosages may be determined by clinical trials.

Therapeutic Applications

As it is already disclosed elsewhere in the present specification, AMHRII-binding agents disclosed herein, which encompass (i) the anti-AMHRII antibodies disclosed herein, (ii) the Antibody Drug Conjugates disclosed herein and (iii) the CAR cells (including the CAR T-cells, the CAR NK cells and the CAR Macrophages) disclosed herein, consist of active ingredients that may be used for preventing or treating AMHRII-expressing lung cancers, especially non-small cell lung cancer (NSCLC) and more precisely a NSCLC selected in a group comprising epidermoid NSCLC, adenocarcinoma NSCLC, large cells NSCLC and squamous cell carcinoma NSCLC and neuroendocrine NSCLC.

Cancer treatment methods that make use of anti-tumor antigen antibodies or anti-tumor antigen CAR cells are well-known from the one skilled in the art.

In some embodiments, cancer patients are tested for determining whether their tumor cells express AMHRII at their surface, before performing a treatment with an AMHRII-binding agent, such as an anti-AMHRII antibody, an anti-AMHRII ADC or an anti-AMHRII CAR T-cells.

Such a preliminary test for detecting membrane expression of AMHRII is preferred for the treatment of lung cancers expressing AMHRII with a low frequency. In contrast, such a preliminary test for detecting membrane expression of AMHRII may not be performed for the treatment of cancers expressing AMHRII at a high frequency, such as illustratively epidermoid NSCLC.

Thus, in some embodiments, this invention relates to an AMHRII-binding agent as specified herein for its use for preventing or treating an individual affected with an AMHRII-positive lung cancer, which includes a a non-small cell lung cancer (NSCLC) and especially a NSCLC selected in a group comprising epidermoid NSCLC, adenocarcinoma NSCLC, large cells NSCLC, squamous cell carcinoma NSCLC, pleiomorphic cell carcinoma NSCLC and neuroendocrine NSCLC.

This invention concerns the use of an AMHRII-binding agent for the preparation of a medicament for preventing or treating an individual affected with an AMHRII-positive lung cancer, which include a lung cancer, which includes a non-small cell lung cancer (NSCLC) and especially a NSCLC selected in a group comprising epidermoid NSCLC, adenocarcinoma NSCLC, large cells NSCLC, squamous cell carcinoma NSCL, pleiomorphic cell carcinoma NSCLC C and neuroendocrine NSCLC.

This invention also pertains to a method for preventing or treating an individual affected with an AMHRII-positive lung cancer, which include a non-small cell lung cancer (NSCLC) and especially a NSCLC selected in a group comprising epidermoid NSCLC, adenocarcinoma NSCLC, large cells NSCLC, squamous cell carcinoma NSCLC, pleiomorphic cell carcinoma NSCLC and neuroendocrine NSCLC, wherein the said method comprises a step of administering to the said individual an anti-AMHRII binding agent.

An individual may be assigned as being an individual affected with an AMHRII-positive cancer by performing a method of detecting cell surface AMHRII protein expression on a lung cancer tissue sample previously obtained from the said individual. Detection of cell surface AMHRII protein expression may be performed according to a variety of methods that are well known from the one skilled in the art. Cell surface AMHRII protein expression detection methods notably encompass immunohistochemistry methods as well as fluorescence activated cell sorting methods that are illustrated in the examples herein.

This invention also relates to a method for determining whether an individual is eligible to a lung cancer treatment with an AMHRII-binding agent, i.e. whether an individual is responsive to a lung cancer treatment with an AMHRII-binding agent, wherein the said method comprises the step of determining whether a lung tumor tissue sample previously obtained from the said individual express the AMHRII protein at the cell surface.

Thus, this invention also relates to a method for determining whether an individual which is affected with a lung cancer, in particular a Non-Small Cell lung Cancer (NSCLC), and especially a NSCLC selected in a group comprising epidermoid NSCLC, adenocarcinoma NSCLC, large cells NSCLC and squamous cell carcinoma NSCLC and neuroendocrine NSCLC, is eligible to a cancer treatment with an AMHRII-binding agent, i.e. is responsive to a cancer treatment with an AMHRII-binding agent, wherein the said method comprises the steps of:
a) determining if cancer cells from the said patient express AMHRII at their membrane, and
b) concluding that the said patient is eligible to a lung cancer treatment with an AMHRII-binding agent, i.e. is responsive to a lung cancer treatment with an AMHRII-binding agent if membrane expression of AMHRII by the said lung cancer cells has been determined at step a).

In preferred embodiments of the said method, it is concluded at step b) that the said patient is eligible (i.e. responsive) to a lung cancer treatment with an AMHRII-binding agent when (i) a AMHRII expression score value is determined at step a) and when (ii) the said AMHRII expression score value is of a threshold score value or more. The AMHRII score value is most preferably calculated by using the formula (I) described elsewhere in the present specification.

Thus, according to preferred embodiments, step a) of the method is performed by a immunohistochemical method, such as shown in the examples herein.

The cancer cells that are used at step a) generally originate from a biopsy tissue sample that has previously been collected from the said cancer patient.

Preferably, step a) is performed by using an anti-AMHRII antibody selected among those specifically described in the present specification, and notably a 3C23K antibody, the AMHRII binding of which may be detected by using a secondary labeled antibody according to well-known antibody detection techniques, such as those disclosed in the examples herein.

Preferably, a patient affected with a lung cancer comprised in the above-listed group of lung cancers is determined as being eligible (i.e. responsive) to a lung cancer treatment with an AMHRII-binding agent when a membranous AMHRII expression score value of 1.0 or more is determined in a cancer cell sample originating from the said cancer patient, when performing a scoring method allowing determination of the E-SCORE value according to the formula (I) below:

E-SCORE=FREQ×AMHRII_LEVEL, wherein
E-SCORE means the membranous AMHRII expression score value for a given cancer cell sample,
FREQ means the frequency of the cells contained in the said lung cancer cell sample for which membrane AMHRII expression is detected, and
AMHRII_LEVEL means the level of membranous expression of AMHRII by the AMHRII-expressing cells contained in the said given lung cancer cell sample.

Thus, the present invention also relates to a method for treating a patient affected with a Non-Small Cell Lung Cancer (NSCLC) wherein the said method comprises the steps of:
a) determining whether a tumor tissue sample previously obtained from the said individual express the AMHRII protein at the cell surface, and
b) treating the said individual with an AMHRII-binding agent if the cell surface expression of AMHRII has been determined at step a).

In most preferred embodiments, AMHRII expression is determined at step a) when the said tumor sample has a membranous AMHRII expression score value “E-SCORE” calculated according to the above-described formula (I) of 1.0 or more, which encompasses an E-SCORE value of 1.5 or more.

In most preferred embodiments of the method above, the said AMHRII-binding agent consists of an anti-AMHRII antibody or fragment thereof as specified herein, or of a CAR cell (e.g. a CAR T-cell or a CAR NK-cell) as specified herein.

In some embodiments, the said AMHRII-binding agent is used as the sole anti-cancer active ingredient.

In some other embodiments, the anti-cancer treatment with the said AMHRII-binding agent also comprises subjecting the said individual to one or more further anti-cancer treatments, which include radiotherapy treatment and chemotherapeutic treatment.

Thus, according to such other embodiments, the anti-cancer treatment with the said AMHRII-binding agent also comprises the administration to the said individual of one or more further anti-cancer active ingredients.

Combination Therapies

As it is shown in the examples herein, efficient anti-lung cancer lung therapies encompass those wherein an anti-AMHRII monoclonal antibody is combined with one or more distinct anti-cancer agent(s). The examples herein illustrate combined therapies against lung cancer, wherein an anti-AMHRII antibody is combined with docetaxel or with a combination of cisplatin and gemcitabine.

An “anticancer agent” is defined as any molecule that can either interfere with the biosynthesis of macromolecules (DNA, RNA, proteins, etc.) or inhibit cellular proliferation, or lead to cell death by apoptosis or cytotoxicity for example. Among the anticancer agents, there may be mentioned alkylating agents, topoisomerase inhibitors and intercalating agents, anti-metabolites, cleaving agents, agents interfering with tubulin, monoclonal antibodies.

A “pharmaceutically acceptable vehicle” refers to a non-toxic material that is compatible with a biological system such as a cell, a cell culture, a tissue or an organism.

According to a particular aspect, the invention relates to a pharmaceutical composition comprising, as active ingredient, in combination with a pharmaceutically acceptable vehicle, an anticancer agent and an antibody binding to AMHR-II, and especially an anti-AMHRII antibody described herein.

In some embodiments, the invention relates to a pharmaceutical composition comprising, as active ingredient, in combination with a pharmaceutically acceptable vehicle, an anticancer agent, and an antibody binding AMHR-II, and especially an anti-AMHRII antibody described herein.

In some embodiments, the invention relates to a pharmaceutical composition comprising, as active ingredient, in combination with a pharmaceutically acceptable vehicle, an anticancer agent, and an antibody binding AMHR-II, in which the anticancer agent is selected in a group comprising docetaxel, cisplatin, gemcitabine and a combination of cisplatin and gemcitabine.

Other anti-cancer agents that may be used in combination with an anti-AMHRII antibody encompass paclitaxel or a platinum salt such as oxaliplatin, cisplatin and carboplatin.

The anticancer agent may also be selected from chemotherapeutic agents other than the platinum salts, small molecules, monoclonal antibodies or else anti-angiogenesis peptibodies.

The chemotherapeutic agents other than the platinum salts include the intercalating agents (blocking of DNA replication and transcription), such as the anthracyclines (doxorubicin, pegylated liposomal doxorubicin), the topoisomerase inhibitors (camptothecin and derivatives: Karenitecin, topotecan, irinotecan), or else SJG-136, the inhibitors of histone deacetylase (vorinostat, belinostat, valproic acid), the alkylating agents (bendamustine, glufosfamide, temozolomide), the anti-mitotic plant alkaloids, such as the taxanes (docetaxel, paclitaxel), the vinca alkaloids (vinorelbine), the epothilones (ZK-Epothilone, ixabepilone), the anti-metabolites (gemcitabine, elacytarabine, capecitabine), the kinesin spindle protein (KSP) inhibitors (ispinesib), trabectedin or else ombrabulin (combretastatin A-4 derivative).

Among the small molecules there are the poly(ADP-ribose)polymerase (PARP) inhibitors: olaparib, iniparib, veliparib, rucaparib, CEP-9722, MK-4827, BMN-673, the kinase inhibitors, such as the tyrosine kinase inhibitors (TKI) among which there may be mentioned the anti-VEGFR molecules (sorafenib, sunitinib, cediranib, vandetanib, pazopanib, BIBF 1120, semaxanib, Cabozantinib, motesanib), the anti-HER2/EGFR molecules (erlotinib, gefitinib, lapatinib), the anti-PDGFR molecules (imatinib, BIBF 1120), the anti-FGFR molecules (BIBF 1120), the aurora kinase/tyrosine kinase inhibitors (ENMD-2076), the Src/Abl kinase inhibitor (Saracatinib), or also Perifosine, Temsirolimus (mTOR inhibitor), alvocidib (cyclin-dependent kinase inhibitor), Volasertib (inhibitor of PLK1 (polo-like kinase 1) protein, LY2606368 (inhibitor of checkpoint kinase 1 (chk 1), GDC-0449 (Hedgehog Pathway Inhibitor), Zibotentan (antagonist of the ETA-receptor), Bortezomib, Carfilzomib (proteasome inhibitor), cytokines such as IL-12, IL-18, IL-21, INF-alpha, INF-gamma.

Among the antibodies, there may be mentioned, the anti-VEGF: bevacizumab, the anti-VEGFR: ramucirumab, the anti-HER2/EGFRs: trastuzumab, pertuzumab, cetuximab, panitumumab, MGAH22, matuzumab, anti-PDGFR alpha: IMC-3G3, the anti-folate receptor: farletuzumab, the anti-CD27: CDX-1127, the anti-CD56: BB-10901, the anti-CD105: TRC105, the anti-CD276: MGA271, the anti-AGS-8: AGS-8M4, the anti-DRS: TRA-8, the anti-HB-EGF: KHK2866, the anti-mesothelins: amatuximab, BAY 94-9343 (immunotoxin), catumaxomab (EpCAM/CD3 bispecific antibody), the anti-IL2R: daclizumab, the anti-IGF-1R: ganitumab, the anti-CTLA-4: ipilimumab, the anti-PD1: nivolumab and pembrolizumab, the anti-CD47: Weissman B6H12 and Hu5F9, Novimmune 5A3M3, INHIBRX 2A1, Frazier VxP037-01LC1 antibodies, the anti-Lewis Y: Hu3S193, SGN-15 (immunotoxin), the anti-CA125: oregovomab, the anti-HGF: rilotumumab, the anti-IL6: siltuximab, the anti-TR2: tigatuzumab, the anti-alpha5 beta1 integrin: volociximab, the anti-HB-EGF: KHK2866. The anti-angiogenesis peptibodies are selected from AMG 386 and CVX-241.

More particularly, it is described herein a pharmaceutical composition comprising, as active ingredient, in combination with a pharmaceutically acceptable vehicle, an anticancer agent, and an antibody binding AMHR-II, in which the anticancer agent is selected in a group comprising docetaxel, cisplatine, gemcitabine and a combination of cisplatine and gemcitabine.

Even more particularly, it is described herein a pharmaceutical composition comprising, as active ingredient, in combination with a pharmaceutically acceptable vehicle, an anticancer agent, and an antibody binding AMHR-II, in which the mutated humanized monoclonal antibody termed 3C23K herein and the anticancer agent is selected in a group comprising docetaxel, cisplatine, gemcitabine and a combination of cisplatine and gemcitabine.

In a particular aspect, it is described herein a pharmaceutical composition comprising, as active ingredient, in combination with a pharmaceutically acceptable vehicle, an anticancer agent, and an antibody binding AMHR-II, in a formulation intended for administration by the intravenous or intraperitoneal route.

In another particular aspect, the invention relates to a composition for use as a medicinal product in the prevention or treatment of a lung cancer, comprising an anticancer agent and an antibody binding AMHR-II, in a formulation intended for administration by the intravenous or intraperitoneal route.

In another particular aspect, the invention relates to a composition for use as a medicinal product in the prevention or treatment of a lung cancer, comprising an anticancer agent and an antibody binding AMHR-II, the monoclonal antibody and the anticancer agent being intended for separate, simultaneous or sequential administration.

The antibody and the anticancer agent may be combined within one and the same pharmaceutical composition, or may be used in the form of separate pharmaceutical compositions, which may be administered simultaneously or sequentially. In particular, the products may be administered separately, namely either concomitantly, or independently, for example with a time gap.

More particularly, the invention relates to a composition for use as a medicinal product in the prevention or treatment of a lung cancer, comprising an anticancer agent and an antibody binding AMHR-II, in which the antibody and the anticancer agent are combined within the same pharmaceutical composition.

According to another particular aspect, the invention relates to a composition for use as a medicinal product in the prevention or treatment of a lung cancer, comprising an anticancer agent and an antibody binding AMHR-II, in which the therapeutically effective quantity of the anti-AMHRII antibody administered to a patient is in a range from about 0.07 mg to about 35 000 mg, preferably from about 0.7 mg to about 7000 mg, preferably from about 0.7 mg to about 1400 mg, preferably from about 0.7 mg to about 700 mg, and more preferably from about 0.7 mg to about 70 mg.

According to another particular aspect, the invention relates to a composition for use as a medicinal product in the prevention or treatment of a lung cancer, comprising an anticancer agent and an antibody binding AMHR-II, in which the therapeutically effective quantity of anticancer agent administered to a patient is in a range from about 10 mg to about 700 mg, preferably in a range from about 20 mg to about 350 mg, and preferably about 110 mg.

According to another particular aspect, the invention relates to a composition for use as a medicinal product in the prevention or treatment of a lung cancer, comprising an anticancer agent and an antibody binding AMHR-II, in which the therapeutically effective quantity of antibody administered to a patient is about 70 mg and the dose of anticancer agent administered to the patient is about 110 mg.

In a preferred embodiment, the dosage of anticancer agent, in particular docetaxel, or the combination of cisplatine and gemcitabine, is in a range from about 0.01 mg/kg to about 500 mg/kg, for example 0.1 mg/kg to 300 mg/kg, or from about 0.1 mg to 20 g per day.

As a variant, a higher initial loading dose, followed by one or more lower doses may also be administered. In another variant, an initial loading dose that is not so high, followed by one or more higher doses may also be administered.

In a particular embodiment, the anti-AMHR-II antibody and the anti-cancer agent may be used in an antibody/anti-cancer agent weight ratio in a range from about 10/1 to about 0.01/1, in particular from about 10/1 to about 0.05/1, or from about 5/1 to about 0.1/1.

Illustratively, the anti-AMHRII antibody and docetaxel may be used in an antibody/docetaxel weight ratio of 1/1, as shown in the examples herein.

Still illustratively, the anti-AMHRII antibody and cisplatine may be used in an antibody/cisplatine weight ratio of 4/1, as shown in the examples herein.

Yet illustratively, the anti-AMHRII antibody and gemcitabine may be used in an antibody/gemcitabine weight ratio of 0.2/1, as shown in the examples herein.

The invention further describes a product comprising an antibody binding the human anti-Müllerian hormone type II receptor (AMHR-II) and an anticancer agent, in the form of a combined preparation, for simultaneous, sequential or separate use as a medicinal product intended for preventing or treating an AMHRII-expressing lung cancer.

An AMHRII-binding agent as disclosed herein, and especially an anti-AMHRII antibody disclosed herein, may be administered in various ways, which include oral administration, subcutaneous administration, and intravenous administration.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, duration of progression free survival (PFS), the response rates (RR), duration of response, and/or quality of life.

Therapeutic formulations of the agents (e.g., antibodies) used in accordance with the invention are prepared for storage by mixing an antibody having the desired degree of purity with optional pharmaceutically acceptable carriers, excipients or stabilizers {Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes {e.g. Zn-protein complexes); and/or non-ionic surfactants such as TWEEN™ PLURONICS™ or polyethylene glycol (PEG).

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration may be sterile. This is readily accomplished by filtration through sterile filtration membranes.

A pharmaceutical composition as described herein may be administered by any suitable administration route, for example by the parenteral, oral, sublingual, vaginal, rectal, or transdermal route, preferably by intravenous, subcutaneous or intradermal injection. Intramuscular, intraperitoneal, intrasynovial, intrathecal or intratumoral injection is also possible. The injections may be carried out in the form of a bolus, or by continuous infusion. When the antibody composition and the composition of anticancer agent are administered separately, these compositions may be in an identical or different form of administration.

The preparations for parenteral administration may include sterile aqueous or non-aqueous solutions, suspensions or emulsions. Examples of non-aqueous solvents are propylene glycol, polyethylene glycol, vegetable oils, such as olive oil, or injectable organic esters such as ethyl oleate. Aqueous vehicles comprise water, alcohol/water solutions, and emulsions or suspensions.

The pharmaceutical compositions as described herein advantageously comprise one or more pharmaceutically acceptable excipients or vehicles. There may be mentioned for example saline, physiological, isotonic, buffered solutions, etc., compatible with pharmaceutical use and known to a person skilled in the art. The compositions may contain one or more agents or vehicles selected from dispersants, solubilizers, stabilizers, preservatives, etc. Agents or vehicles usable in formulations (liquid and/or injectable and/or solid) are in particular methylcellulose, hydroxymethylcellulose, carboxymethylcellulose, polysorbate 80, mannitol, gelatin, lactose, vegetable oils, acacia, etc. The compositions may be formulated in the form of injectable suspensions, gels, oils, tablets, suppositories, powders, hard gelatine capsules, soft capsules, etc.

According to a particular aspect, the invention relates to a pharmaceutical composition comprising, as active ingredient, in combination with a pharmaceutically acceptable vehicle, an anticancer agent and an anti-AMHRII antibody, in which the therapeutically effective quantity of antibody administered to a patient is in a range from about 0.07 mg to about 35000 mg, preferably from about 0.7 mg to about 7000 mg, preferably from about 0.7 mg to about 1400 mg, preferably from about 0.7 mg to about 700 mg, and more preferably from about 0.7 mg to about 70 mg.

The dosage of the active ingredient depends in particular on the administration method, and is easily determined by a person skilled in the art. A therapeutically effective quantity (unit dose) of antibody may vary from 0.01 mg/kg to 500 mg/kg, preferably from 0.1 mg/kg to 500 mg/kg, preferably from 0.1 mg/kg to 100 mg/kg, preferably from 0.1 mg/kg to 20 mg/kg, preferably from 0.1 mg/kg to 10 mg/kg, and more preferably from 1 mg/kg to 10 mg/kg, in one or more weekly administrations, for several weeks or months. The effective unit dose may therefore easily be deduced from a dose calculated for an “average” patient with a weight of 70 kg.

According to another particular aspect, the invention relates to a pharmaceutical composition comprising, as active ingredient, in combination with a pharmaceutically acceptable vehicle, an anticancer agent and an anti-AMHRII antibody, in which the therapeutically effective quantity of anticancer agent administered to a patient is in a range from about 10 mg to about 700 mg, preferably in a range from about 20 mg to about 350 mg, and preferably is about 110 mg.

The dosage of the anticancer agent depends in particular on the administration method, and is easily determined by a person skilled in the art. A therapeutically effective quantity (unit dose) may vary from 0.2 mg/m² to 10 g/m², preferably from 0.2 mg/m² to 1 g/m², preferably from 2 mg/m² to 1 g/m², preferably from 20 mg/m² to 1 g/m², and more preferably from 20 mg/m² to 0.5 g/m², in one or more weekly administrations, for several weeks or months. The effective unit dose may therefore be deduced from a dose calculated for an “average” patient whose body surface area is about 1.8 m².

According to an even more particular aspect, the invention relates to a pharmaceutical composition comprising, as active ingredient, in combination with a pharmaceutically acceptable vehicle, an anticancer agent and an anti-AMHRII antibody, in which the therapeutically effective quantity of anticancer agent administered to a patient is about 110 mg, and the therapeutically effective quantity of antibody administered to the patient is about 70 mg.

The invention also describes a composition comprising an anticancer agent and an anti-AMHRII antibody binding the human anti-Müllerian hormone type II receptor (AMHR-II), for use as a medicinal product in the prevention or treatment of an AMHRII-expressing lung cancer.

The present invention is further illustrated by, without in any way being limited to, the examples below.

### EXAMPLES

### Example 1: Differential AMHRII Gene Expression and AMHRII Protein Expression

A. Materials and Methods

A.1. Cell Lines and Cultures

The COV434 WT cell line (ECACC N^{o} 07071909) was maintained in DMEM/GlutaMax (Gibco) supplemented with 10% FBS, penicillin 100 U/ml and Streptomycin 100 μg/ml. Geneticin (Gibco) at 400 μg/ml was added for the COV434 MISRII transfected cell line. The erythroleukemia K562 cell line (ATCC® CCL-243™) was cultivated in suspension in IMDM medium (Sigma-Aldrich) supplemented with 10% FBS and penicillin/Streptomycin and maintained at a density between 1×10⁵ and 1×10⁶ cells/ml in T75 flasks. The OV90 cell line (ATCC® CRL-11732™, ovary serous adenocarcinoma) was cultivated in a mixture 1:1 of MCDB 105 medium (Sigma-Aldrich) containing a final concentration of 1.5 g/l sodium bicarbonate and medium 199 (Sigma-Aldrich) containing a final concentration of 2.2 g/l sodium bicarbonate supplemented with 15% FBS and penicillin/Streptomycin. The NCI-H295R cell line (adrenocortical carcinoma, ATCC® CRL-2128™) was maintained in DMEM:F12 medium (Sigma-Aldrich) supplemented with iTS⁺Premix (Corning), 2.5% Nu-Serum (Falcon) and penicillin/Streptomycin. Cells were grown at 37° C. in a humidified atmosphere with 8% CO2 and medium was replaced one or twice a week depending the cell lines.

A.2. Relative Quantification of AMHR2 mRNA by RT-qPCR

Extraction of RNA. Total RNA from 1-5×10⁶ cells pellet was prepared using Trizol® Plus RNA Purification Kit (Ambion) according to the manufacturer's instructions. Briefly, after phenol/chloroform extraction, RNA of lysed cells was adsorbed on silica matrix, DNAse treated, then washed and eluted with 30 μl of RNAse free water. RNA concentrations and quality were assessed with spectrophotometer (NanoDrop, ThermoFisher Scientific).

cDNA synthesis. RNA (1 μg) was reverse transcribed using Maxima H Minus First Strand cDNA Synthesis Kit (Ambion) and oligo-dT primers by incubation 10 min at 25° C. for priming and 15 min at 50° C. for reverse transcription followed by 5 min at 85° C. for reverse transcriptase inactivation.

Quantitative PCR. Quantitative PCR was performed in Light Cycler 480 (Roche) in 96-wells microplates using Luminaris Color HiGreen qPCR Master Mix (Ambion) in a final volume of 20 μl. The following primers were used: for AMHR2, Forward 5′-TCTGGATGGCACTGGTGCTG-3′ (SEQ ID NO. 71) and Reverse 5′-AGCAGGGCCAAGATGATGCT-3′ (SEQ ID NO. 72), for TBP, Forward 5′-TGCACAGGAGCCAAGAGTGAA-3′(SEQ ID NO. 73) and Reverse 5′-CACATCACAGCTCCCCACCA-3′ (SEQ ID NO. 74). Amplications were performed using cDNA template (100 ng equivalent RNA) and the following protocol: UDG pretreatment 2 min at 50° C., denaturation 10 min at 95° C. followed by 40 cycles of 15 s at 95° C./30 s at 60° C./30 s at 70° C. A melting curves analysis was performed at the end of each experiments to control the absence of genomic DNA and dimer primer. Each cDNA samples and controls (“no template sample” and “no reverse transcript RNA”) were tested in duplicate. The mean values of Cycle Threshold (Ct) were calculated and the AMHR2 relative quantification (RQ) was expressed as 2^{-ΔΔCt} where ΔΔCt=ΔCtₛₐₘₚₗₑ−ΔCt_{calibrator} and ΔCt=Ct_{AMHR2}−Ct_{TBP}. HCT116 sample was used as calibrator and TBP as housekeeping gene for normalization.

Table 2 below depicts the AMHRII expression level in the tested cell lines using the Q-PCR method described above.

TABLE 2

| | Mean Ct | Mean Ct | |
|---|---|---|---|
| Cell line | amhr2 | TBP | RQ |
| HCT116 | 34.27 | 22.25 | 1 |
| COV434 WT | 31.34 | 22.82 | 11.3 |
| K562 | 25.31 | 21.36 | 268.7 |
| NCI-H295R | 26.16 | 22.83 | 413.0 |
| OV90 | 25.65 | 22.67 | 526.4 |

A.3. Evaluation of Membrane AMHR2 Expression by Flow Cytometry Analysis.

For Fluorescent-Activated Cell Sorting (FACS) analysis, 4×10⁵ cells were incubated with 25 μg/ml of 3C23K for 30 min at 4° C. After washes with PBS-BSA2%, the primary antibody was detected by an anti-species secondary antibody conjugated to a fluorophore. The 3C23K was detected by an anti-human F(ab′)₂ conjugated to Phycoerythrin (1:1000, Beckman-Coulter, IM0550). After washes with PBS, FACS analysis of the resuspended cells was realized in the FL2 channel of the BD Accuri™ C6 flow cytometer (BD Bioscience).

B. Results

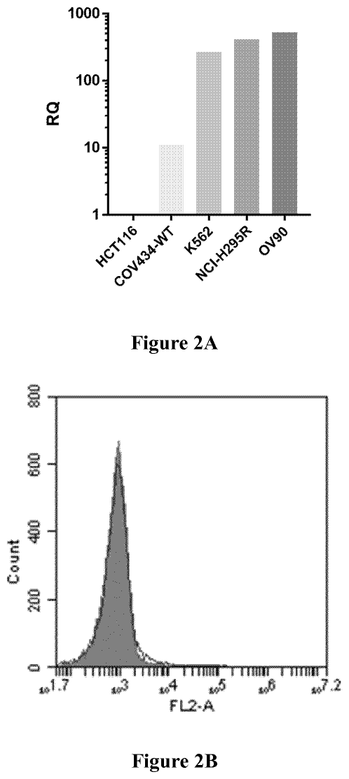

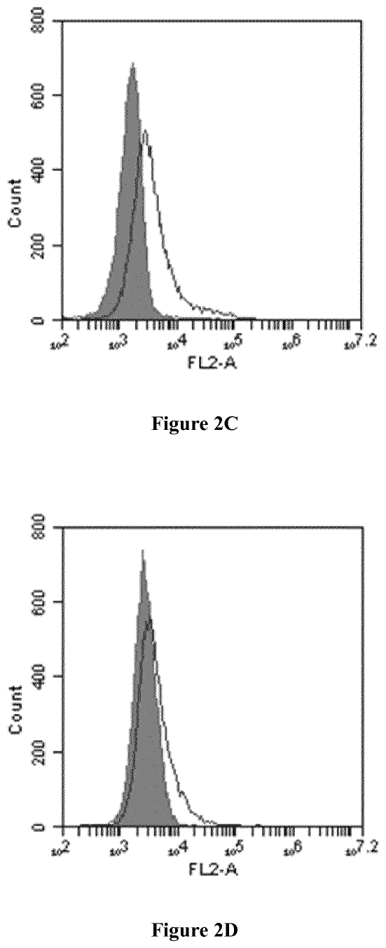

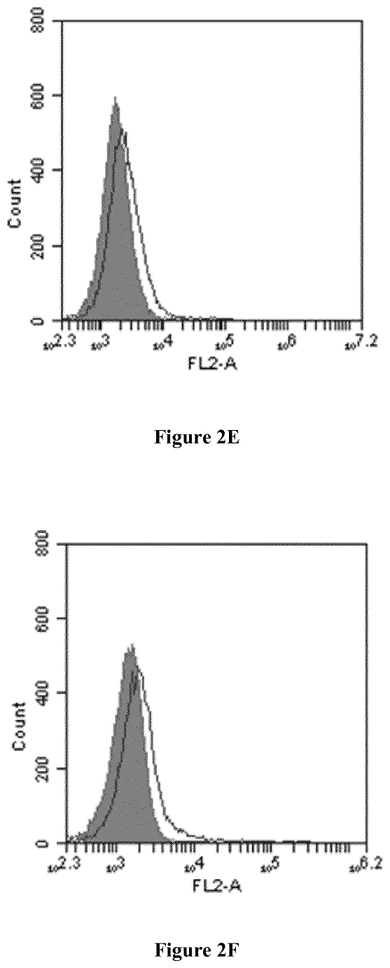

The results are depicted in FIG. 2. The results showed that the recombinant cell line COV434-WT (about 3% of the AMHRII gene expression level measured for the cell line NCI-H295R) although the COV434-WT cell line has a significative membrane expression level of human AMHRII protein.

These results showed that there is strictly no correlation between AMHRII gene expression and membrane AMHRII protein expression.

### Example 2: AMHRII Expression in Lung Cancers (Human Tumor Samples)

A. Materials and Methods

A.1. Objective

Immunohistochemical study of human cancer cells xenografts in mice (PDXs) for detecting anti-Müllerian hormone receptor type 2 (AMHR2) expression using a biotinylated 3C23K monoclonal antibody.

A.2. Protocol and Methodology
The cell lines: fixed in formaldehyde acetic acid alcohol (AFA) with constitution of cellblocks
Human Tumors: fixation in formalin for external samples and in AFA for slides from Curie Institute
Immunohistochemistry (IHC) technique was possible after dewaxing samples and unmasking at pH9 (microwave EZ Retriever 15′ at 90° C., followed by cooling during 20′).
Anti-Mullerian Hormone Receptor Type II detection by immunoperoxidase technique and DAB chromogenic substrate revelation.
After blocking endogenous peroxydase activity, the slides were incubated with diluted biotinylated primary antibody (1/800, 8 μg/mL) for 90 minutes at room temperature. The tissue sections were then washed with PBS and incubated with avidin/biotin ABC [Vector] complex for 30 minutes. Immunoreactive signals were detected using DAB substrate solution (DAB+ Substrate buffer/Liquid DAB+ chromogen, 10 minutes incubation). Finally, the sections were lightly counterstained with Mayer's Hematoxylin (Lillie's Modification).
Negative controls were obtained by substitution of the primary antibodies with isotype control immunoglobulin (R565) or with antibody diluent alone (negative buffer control) in the immunohistochemical staining procedure.
Positive controls were obtained by using AMHR2-transfected COV434 cells and human granulosa tumor samples
After processing, sections were observed by digitalization via Philips IMS. All specimens were scored independently by 2 pathologists.
Localization of the labeling was detailed: cytoplasmic and/or membranous.
Intensity was classified as unequivocal brown labeling of tumor cell membrane and/or cytoplasm through the following scoring system: intensity of the labeling was defined as 0 for negative, 1 for weak, 2 for moderate, and 3 for strong as shown in the COV434 positive control.
Frequency was defined as a percentage of cells expressing AMHRII. Necrotic areas were excluded from analysis. The Global Histological score was established by using frequency x mean of intensity scores (0 to 3) cumulating membranous and cytoplasmic expression.
All slides were duly stored.

B. Results

The results of AMHRII membrane expression by various primary human lung cancer cells are also depicted in Table 3, wherein the AMHRII expression score is represented for a panel of distinct lung cancer samples.

TABLE 3
AMHRII expression by human lung cancer tissue samples

| | Percentage of | Number of |
|---|---|---|
| Tumor type | positive samples | samples tested |
| SCLC | 0% | 2 |
| NSCLC (neuroendocrine) | 1.2% | 78 |
| NSCLC (acinar-type) | 0% | 2 |
| NSCLC (epidermoïd) | 100% | 4 |
| NSCLC (squamous cell carcinoma) | 35% | 14 |
| NSCLC (adenocarcinoma) | 45.8% | 24 |
| NSCLC (large cells) | 33% | 9 |

The results showed that AMHRII is expressed at the cell surface in a plurality of lung human cancer samples, especially by NSCLC-derived tumor samples and more precisely by tumor samples originating from patients affected with a NSCLC selected in the group consisting of epidermoid NSCLC, adenocarcinoma NSCLC, large cells NSCLC and squamous cell carcinoma NSCLC and neuroendocrine NSCLC.

### Example 3: AMHRII Expression in Lung Cancers

A. Materials and Methods

A.1. Objective

A study of human lung cancer cells issued either from patient derived xenografts (PDXs) or fresh human tumor samples was initiated for detecting anti-Müllerian hormone receptor type 2 (AMHR2) expression using a biotinylated 3C23K monoclonal antibody.

A.2. AMHRII Membrane Expression Analysis by Flow Cytometry

Preparation of Cells for Analysis
Tissues were dissected within 1 h of surgery, minced into 1-mm2 fragments and washed in RPMI containing penicillin (10%), streptomycin (10%) and gentamycin (0.1 mg/mL; Sigma-Aldrich).
Tissue fragments were digested for 2-4 h with collagenase and DNAse (2 mg/mL; Sigma-Aldrich) with rapid shaking at 37 C.
Mucus and large debris were removed by filtration through a 40-lm cell strainer.
Viable cells were obtained by Ficoll gradient centrifugation.

The quantitation of AMHRII binding sites on resuspended tumor cells was performed using The Quantum™ Simply Cellular (Bangs Laboratory) according to the manufacturer's instructions:
Briefly, the four microbeads populations labeled with a different calibrated amount of mouse anti-human IgG specific for the Fc portion of human IgG antibodies were stained with the AlexaFluor488-conjugated anti-AMHRII 3C23K. In FACS tubes, one drop of each vial in the kit is added to 50 μl of PBS 1×:
   1—Beads B (blank)
   2—Beads 1+3C23K-AF 10 μg/mL
   3—Beads 2+3C23K-AF 10 μg/mL
   4—Beads 3+3C23K-AF 10 μg/mL
   5—Beads 4+3C23K-AF 10 μg/mL (the concentration could be increased to 25 μg/ml if necessary)
Each bead population binds varying amounts of the AlexaFluor488-conjugated anti-AMHRII 3C23K, producing a corresponding intensity of fluorescence, which is analyzed on a FACS Canto II cytometer (BD).
A calibration curve was generated by plotting the mean fluorescence intensity of each bead population versus its assigned Antibody Binding Capacity (ABC).

Cells were usually stained in eppendorf tubes 1.5 ml.
All centrifugation steps were done at 4° C.
All incubation steps were done at 4° C. to avoid antibody internalization.
3.5 Million Cells (trypsinized COV434-MISRII or freshly dissociated tumor cells) were centrifuged at 200-300 g for 5 min and were washed one time with PBS (500 μl per tube)
Wash with ice cold PBS/2% FBS (200-300 g for 3 min) and resuspend in 700 μl of PBS 1× and distribute 100 μl by FACS tube for the conditions described in Table 4 below:

TABLE 4

| | |
|---|---|
| COV434-MISRII | Fresh tumor cells |

No antibody
R565-AF (isotype control) 10 μg/mL

3C23K-AF 1 ng/mL
3C23K-AF 10 ng/mL
3C23K-AF 100 ng/mL
3C23K-AF 1 μg/mL

3C23K-AF 10 μg/mL (and up to 25 μg/ml when necessary)

Incubate with antibody 3C23K-AF488 in PBS/1% FBS for 30 min at 4° C.
Wash in PBS/2% BSA two times (200-300 g for 3 min)
Wash in PBS two times (200-300 g for 3 min)
Add 300-400 μl PBS and analyze on FACS as soon as possible

This protocol does not comprise any fixation step for extracellular staining to maintain the integrity of the membrane. Consequently, only membrane AMHRII is detected

A.3. Immunohistochemistry: Protocol and Methodology
The cell lines: fixed in formaldehyde acetic acid alcohol (AFA) with constitution of cellblocks
Human Tumors: fixation in formalin for external samples and in AFA for slides from Curie Institute
Immunohistochemistry (IHC) technique was possible after dewaxing samples and unmasking at pH9 (microwave EZ Retriever 15′ at 90° C., followed by cooling during 20′).
Anti-Mullerian Hormone Receptor Type II detection by immunoperoxidase technique and DAB chromogenic substrate revelation.
After blocking endogenous peroxydase activity, the slides were incubated with diluted biotinylated primary antibody (1/800, 8 μg/mL) for 90 minutes at room temperature. The tissue sections were then washed with PBS and incubated with avidin/biotin ABC [Vector] complex for 30 minutes. Immunoreactive signals were detected using DAB substrate solution (DAB+ Substrate buffer/Liquid DAB+ chromogen, 10 minutes incubation). Finally, the sections were lightly counterstained with Mayer's Hematoxylin (Lillie's Modification).
Negative controls were obtained by substitution of the primary antibodies with isotype control immunoglobulin (R565) or with antibody diluent alone (negative buffer control) in the immunohistochemical staining procedure.
Positive controls were obtained by using AMHR2-transfected COV434 cells and human granulosa tumor samples
After processing, sections were observed by digitalization via Philips IMS. All specimens were scored independently by 2 pathologists.
Localization of the labeling was detailed: cytoplasmic and/or membranous.
Intensity was classified as unequivocal brown labeling of tumor cell membrane and/or cytoplasm through the following scoring system: intensity of the labeling was defined as 0 for negative, 1 for weak, 2 for moderate, and 3 for strong as shown in the COV434 positive control.
Frequency was defined as a percentage of cells expressing AMHRII. Necrotic areas were excluded from analysis. The Global Histological score was established by using frequency x mean of intensity scores (0 to 3) cumulating membranous and cytoplasmic expression.
All slides were duly stored.

B. Results

a) Controls
The negative control and isotype control were devoid of reactivity on tumor cells.
The positive control sample (COV434 AMHRII amplified) showed a diffuse immunostaining of cells (intensity score: 3). The labeling was homogeneous (frequency score: 100%) with cytoplasmic and membranous localization.
The positive Granulosa control sample showed a strong immunostaining of tumor cells (intensity score 3). The labeling was homogeneous (frequency score: 100%) with cytoplasmic and membranous localization.

b) AMHRII Expression of Patient-Derived Xenografts (PDX) Samples as Assessed by IHC.

It is important to notice that membranous expression of AMHR2 seems to be underestimated when samples are fixed in formalin in comparison to samples processed in AFA.

The results of AMHRII membrane expression by various human tumors xenografted in mice are depicted in Table 5, wherein the AMHRII expression score is represented for a panel of distinct cancer cell types.

Part of the results of AMHRII expression by human tumor xenografts are summarized in Table 5 hereunder.

TABLE 5
AMHRII expression in human tumor xenografts

| | Percentage of | Number of |
|---|---|---|
| Tumor type | positive PDXs | PDXs tested |
| SCLC | 0% | 13 |
| NSCLC (non-specified subtype) | 15.4% | 13 |
| NSCLC (epidermoïd) | 26.9% | 26 |
| NSCLC (adenocarcinoma) | 7.7% | 39 |
| NSCLC (large cells) | 40% | 10 |

The results showed that AMHRII is expressed at the cell surface in a plurality of lung human cancer xenografts, especially by NSCLC-derived tumor samples and more precisely by tumor samples originating from patients affected with a NSCLC selected in the group consisting of epidermoid NSCLC, adenocarcinoma NSCLC, large cells NSCLC and some NSCLC not yet identified sub-types.

c) AMHRII Expression of Patient-Derived Xenografts (PDX) Samples as Assessed by Flow Cytometry (FACS)

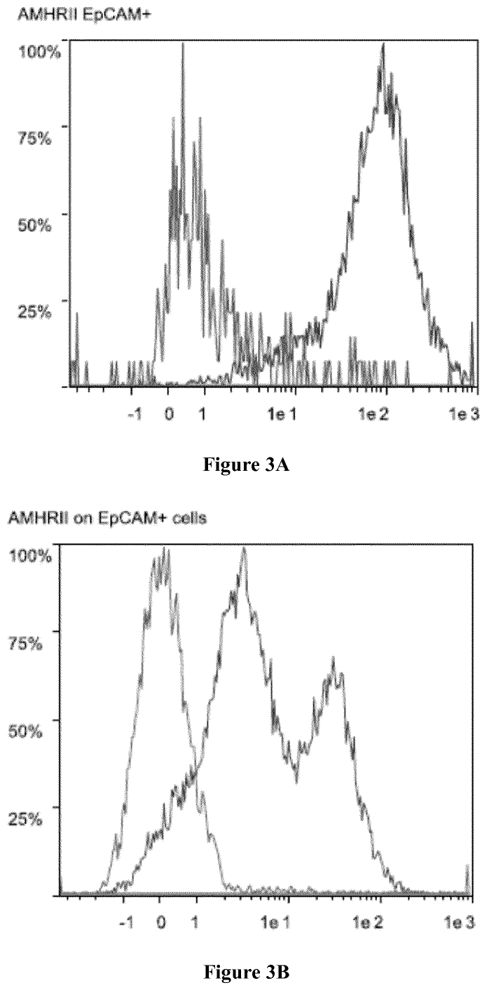

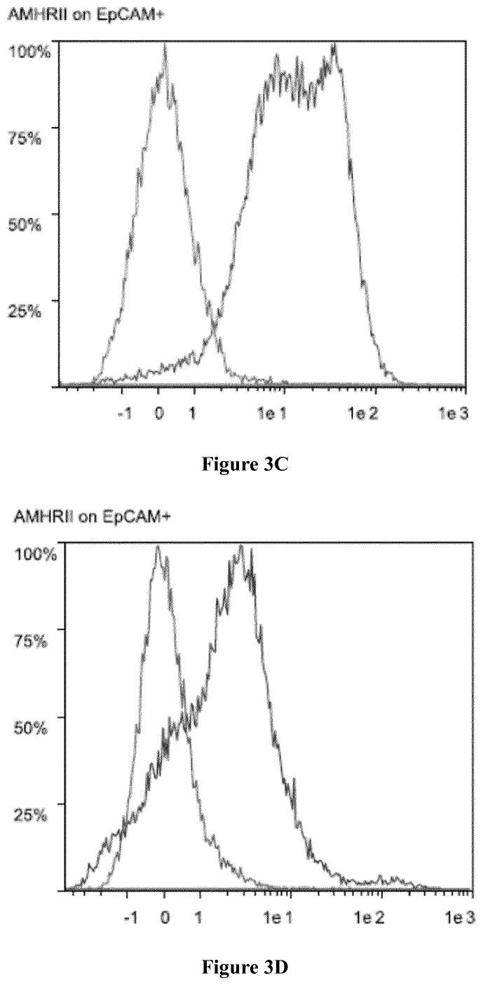

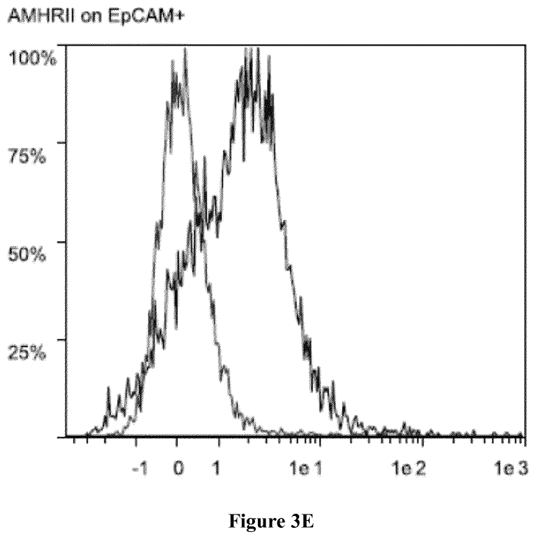

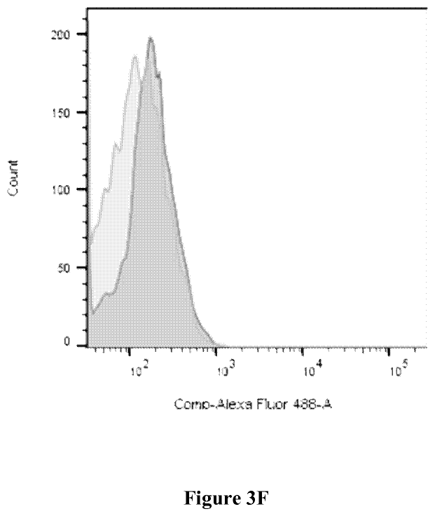

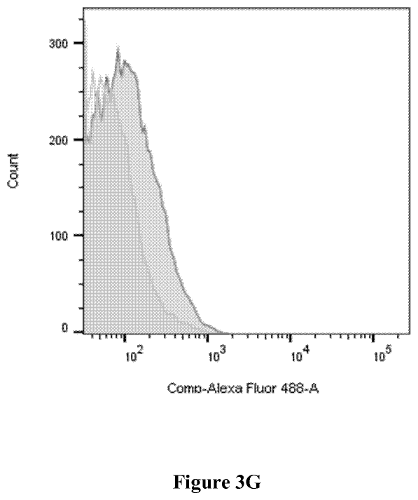

The results depicted in FIGS. 3A to 3E show that AMHRII is expressed at the membrane of tumor cells originating from lung cancer patient derived xenografts, irrespective of the kind of lung cancer which is considered. The results depicted in FIGS. 3A to 3E show that membrane protein expression of AMHRII is found in squamous cell lung carcinoma (FIGS. 3A, 3C, 3D), large cell lung carcinoma (FIG. 3B) and pleiomorphic cell lung carcinoma (FIG. 3E).

Further, for the same lung cancer cells, (i) the number of AMHRII per cell as well as (ii) the percentage of AMHRII cancer cells in the same tested samples were measured. The results are depicted in Table 6 hereunder.

TABLE 6
FACS Analysis of AMHRII expression in human lung cancer
cells obtained from patient derived xenografts

| | | Number of | Percentage of | |
|---|---|---|---|---|
| | | AMHRII | AMHRII | |
| | Ref | receptors per | positive cells | Positive/ |
| Histological type | number | cell | (in %) | negative |
| Squamous cell | Lu7860 | 519.000 | 100 | + |
| carcinoma | | | | |
| Large cell | Lu7166 | 51.000 | 70 | + |
| carcinoma | | | | |
| Squamous cell | Lu7298 | 150.000 | 87 | + |
| carcinoma | | | | |
| Squamous cell | Lu7414 | 61.000 | 63 | + |
| carcinoma | | | | |
| Pleiomorphic cell | Lu7558 | 46.000 | 35 | + |
| carcinoma | | | | |

In Table 6, AMHRII expression was assessed, in each tumor sample, by (i) determining the mean number of AMHRII proteins present at the tumor cell membrane and by (ii) determining the percentage of membranous AMHRII positive cells in the tumor sample. Indication of whether the corresponding tumor sample is set to be “positive” or “negative” is presented in the left column of Table 6. Indication “positive” means that the tumor cells of the lung cancer patient express AMHRII significantly at their membrane. Indication “negative” means that AMHRII is not detected significantly at the tumor cell membrane.

The results of Table 6 show that all tumor samples expressed membranous AMHRII, albeit at various expression levels.

d) AMHRII Expression of Fresh Human Tumor Samples as Assessed by Flow Cytometry (FACS)

The results depicted in FIGS. 3F and 3G show that AMHRII is expressed at the membrane of tumor cells originating from a human NSCLC surgically resected (FIG. 3G) while AMHRII is not expressed at the membrane of the cells originating from the healthy margin issued from the same patient (FIG. 3F).

e) Conclusions

AMHR2 protein expression was confirmed for lung cancer PDX models positive for AMHR2 transcription. These PDXs were adapted from lung (IC8LC10 and SC131 cancers. Levels of expression were moderate but significant, characterized by global score of 1 to 1.5. These data suggest that other than gynecological cancer could express AMHR2.

These models could be used for characterizing anti-AMHR2 therapies in the future.

### Example 4: In Vivo Efficacy of Anti-AMHRII Antibodies Against AMHRII-Expressing Lung Cancer

1. Objective Summary

To analyze the antitumor efficacy of Gamamab's test compound GM102 (also termed 3C23K antibody herein) used as single agent or in combination either with docetaxel or the combination cisplatin/gemcitabine in the SC131 patient-derived non-small-cell lung xenograft model, developed in immunodeficient female mice.

2. Methods

Fifty-four (54) mice with a subcutaneously growing SC131 tumor (P22.1.3/0) between 62.5 and 220.5 mm³ were allocated to treatment when the mean and median tumor volume reached 130.76 and 126.00 mm³ respectively.

The efficacy study XTS-1526 consisted in 6 groups of 9 mice each:
In group 1, vehicle was dosed at 5 ml/kg, i.v. 2 qwk×3;
In group 2, GM102 was dosed at 20 mg/kg, i.v. 2 qwk×3;
In group 3, docetaxel was dosed at 20 mg/kg, slow i.v. once on D0;
In group 4, GM102 was dosed at 20 mg/kg, i.v. 2 qwk×1 or 2 in combination with docetaxel at 20 mg/kg, slow i.v. once on D0;
In group 5, cisplatin was dosed at 5 mg/kg combined with gemcitabine at 100 mg/kg, both i.p. qwk×2 or 3;
In group 6, GM102 was dosed at 20 mg/kg, i.v. 2 qwk×1 or 2 with the combination cisplatin at 5 mg/kg and gemcitabine at 100 mg/kg, both i.p. qwk×1 or 2.

From non-included efficacy study mice, 2 groups including 8 mice per group were tested:
In group 7, GM102 was dosed at 20 mg/kg, i.v. 2 qwk×3 in combination with cisplatin at 5 mg/kg, i.p. qwk×3;
In group 8, GM102 was dosed at 20 mg/kg, i.v. 2 qwk×3 in combination with gemcitabine at 100 mg/kg, i.p. qwk×3.

Tumors were measured and mice were weighed three times a week during the experimental period. Fresh tumor samples were collected from 3 mice per group without extra-dose on D28 (for inclusion 2 and 3) or D31 (for inclusion 1) for snap-frozen tissue and formalin fixed samples. Only snap-frozen tissue were forward for subsequent analyse. The formalin fixed samples were discarded after sampling.

3. Aim of the Study

The experiment described in this report aimed at determining antitumor efficacy of one Gamamabs' test compound, coded GM102, used alone or combined with either docetaxel or the combination cisplatin/gemcitabine in the SC131 patient-derived non-small-cell lung xenograft model.

Test Item: GM102 (Also Termed 3C23K Herein)

The anti-AMHR2 product, GM102 is a humanized mAb directed against the receptor of the anti-Müllerian hormone (AMHR2), alternatively known as Müllerian Inhibiting Substance Receptor II (MISRII). AMHR2 is present during intra-uterine period at the level of internal sexual female organ precursors (Müllerian tractus), and is restricted to ovary (Granulosa cells) and testis (Leydig cells) during adulthood. AMHR2 is also expressed in about 65% of gynecologic cancers such as ovary and endometrium (Bakkum J N, Gynecol Oncol, 2007; Sahli I, Biochem, 2004; Anttonen M, Lab Invest, 2011; Song J Y, Int J. Oncol, 2009).

The GM102 antibody has been shown to display antitumor efficacy in mouse xenograft models using AMHR2-transfected human tumor cell lines. This efficacy has been documented to rely on engagement of immune effector cells triggered by the enabling optimized antibody at the level of the tumor. In addition, GM102 efficacy has been shown to be synergistic with carboplatin and paclitaxel, the major chemotherapeutic agents used in ovarian cancer (Jacquet A., Cancer Res, 2012).

The Human Tumor Xenograft Models

Human tumor samples of various histological origins were obtained with informed consent from patients treated at cancer centers and established as transplantable xenografts in immunodeficient mice. The grafted samples are residual material from primary tumors or metastases obtained before or after treatment. These patient-derived xenograft (PDX) models have been established without prior in vitro culture and have been studied for histology, cytogenetics, genetic and other biological markers, and for their response to standard-of-care (SOC) therapies.

The SC131 tumor model has been derived from a skin metastasis of non-small cell lung cancer with mutated EGFR (R451F) and Kras (G12V), and wild-type TP53 and PTEN.

SC131 is low responder to docetaxel and to the combination cisplatin/gemcitabine, and no responder to the other agents tested (data obtained on swiss nude mice).

SC131 tumor model takes about 17 days to obtain the maximum of tumors in the range 60 to 200 mm³ and 35 to 40 days to reach 2000 mm³ from implantation day.

SC131 shows cachectic properties.

4. Materials

4.1. Animals and Maintenance Conditions

Outbred athymic (nu/nu) female mice («HSD: Athymic Nude-Foxn1^{nu}») weighing 18-25 grams (ENVIGO, Gannat, France) were allocated to acclimate in the animal facility with access to food and water ad libitum for at least 6 days prior to manipulation (Table 7).

TABLE 7
Animal Characteristics

| | | | | | Age at |
|---|---|---|---|---|---|
| Species | Strain | Supplier | Gender | Weight | reception |
| Mouse | Athymic Nude - | ENVIGO, | Female | 18-25 | 5 weeks |
| (Mus | Foxn1^{nu} | France | | | |
| musculus) | | | | | |

4.2. Statement on Animal Welfare

The authorization to use animals in the CERFE facilities was obtained by The Direction des Services Vétérinaires, Ministère de l'Agriculture et de la Pêche, France (agreement No. B-91-228-107). The animal care and housing are in accordance with French regulatory legislation concerning the protection of laboratory animals.

All experiments were performed in accordance with French legislation concerning the protection of laboratory animals and in accordance with a currently valid license for experiments on vertebrate animals, issued by the French Ministry for Agriculture and Fisheries to Guillaume Lang (No. A-75-1927 dated 15 Apr. 2012; validity: 5 years).

4.3. Animal Husbandry

Mice were housed in groups of a maximum of 7 animals during acclimation period and a maximum of 6 animals during experimental phase. Mice were housed inside individually ventilated cages (IVC) of Polysulfone (PSU) plastic (mm 213 W×362 D×185 H, Allentown, USA) with sterilized and dust-free bedding cobs. Food and water were sterilized. Animals were housed under a light-dark cycle (14-hour circadian cycle of artificial light) and controlled room temperature and humidity.

At request, the environmental conditions were monitored and the data were retained in the Central Animal House Archives.

4.4. Diet and Water Supply

Drinking water was provided ad libitum. Each mouse was offered daily a complete pellet diet (150-SP-25Type, SAFE) throughout the study. The analytical certificate of animal food and water was retained at the CERFE premises.

4.5. Identification of Animals

All animals were weighed before each experiment and identified by a unique pattern for ear punch numbering system.

Each cage was identified by a paper tag indicating: cage number, mice strain and number, tumor code, date of experiment.

4.6. Test Compound and Formulations

The PBS 1× vehicle was prepared by diluting PBS 10× (Sigma PBS 10×, #P5493-1L, batch SLBJ2848) at 1/10 in sterile deionized water. It was stored at 4° C. for treatment aliquots and GM102 dilution for 30 days.

GM102 (3C23K) concentrated aliquots (batch LP01 [R18H2-LP01]) were received on 2016 Jul. 7 (4 vials of 5 ml at 10.1 mg/ml) and were stored at 4° C. On each dosing day, stock solution was diluted in cold PBS 1× to obtain the 2 mg/ml working solution. This solution was kept on ice or at 4° C. and protected from light until treatment, then the vial was kept at room temperature during the injection. The remaining working solution after treatment was discarded.

Docetaxel (Taxotere®, Sanofi, batch 6F255A—Exp: 03-2018) stock solution at 10 mg/ml has to be diluted, before each dosing, with 0.9% NaCl at 1/5 to obtain a working concentration of 2 mg/ml. The stock solution is stable for one month after reconstitution at 4° C. and protected from light.

Cisplatin (Cisplatin-Teva, batch 15A30MF—Exp: 01-2017) stock solution at 0.5 mg/ml was ready-to-use. This solution was kept at room temperature and protected from light until the supplier expiration date.

Gemcitabine (Gemzar®, Lilly, batch C442937D, exp: 02-2018) stock solution at 40 mg/ml has to be diluted, before each dosing, with 0.9% NaCl at 1/4 to obtain a working concentration of 10 mg/ml. The stock solution is stable for one month after reconstitution at 4° C. and protected from light.

5. Methods

5.1. Tumorgraft Models Induction

Tumors of the same passage were transplanted subcutaneously onto 3-24 mice (donor mice, passage (n−1)). When these tumors reached 700 to 2000 mm³, donor mice were sacrificed by cervical dislocation, tumors were aseptically excised and dissected. After removing necrotic areas, tumors were cut into fragments measuring approximately 20 mm³ and transferred in culture medium before grafting.

Eighty-nine (89) mice were anaesthetized with 100 mg/kg ketamine hydrochloride (batch 5D92—exp: 03-2017, Virbac) and 10 mg/kg xylazine (batch KP0AX9X, Bayer), and then skin was aseptized with a chlorhexidine solution, incised at the level of the interscapular region, and a 20 mm³ tumor fragment was placed in the subcutaneous tissue. Skin was closed with clips.

All mice from the same experiment were implanted on the same day.

5.2. Treatment Phase

In the XTS-1526 efficacy part, 54 mice with a subcutaneously growing SC131 tumor (P22.1.3/0) between 62.5 and 220.5 mm³ were allocated, according to their tumor volume to give homogenous mean and median tumor volume in each treatment arm. Treatments were randomly attributed to boxes housing up to 5 mice and were initiated 18 days post implantation of the tumor (60% inclusion rate with staggered inclusion). The study was staggered-included with 5 mice included per group first, then 4 mice included per group 2 days later. The study was terminated following 31 days after the start of treatment.

TABLE 8

| Group | Agent | Mean (mm³) | Median (mm³) | SEM |
|---|---|---|---|---|
| 1 | vehicle | 129.28 | 126 | 17.80 |
| 2 | GM102 20 mg/kg | 129.83 | 126 | 13.13 |
| 3 | Docetaxel 20 mg/kg | 137.28 | 126 | 15.38 |
| 4 | GM102 20 mg/kg | 130.33 | 126 | 12.15 |
| | Docetaxel 20 mg/kg | | | |
| 5 | Cisplatin 5 mg/kg | 132.67 | 126 | 17.50 |
| | Gemcitabine 100 | | | |
| | mg/kg | | | |
| 6 | GM102 20 mg/kg | 125.17 | 126 | 16.53 |
| | Cisplatin 5 mg/kg | | | |
| | Gemcitabine 100 | | | |
| | mg/kg | | | |

For the additional groups 7 and 8, tumor size was higher and more heterogeneous. Animals were included 32 days post implantation.

TABLE 9

| Group | Agent | Mean (mm³) | Median (mm³) | SEM |
|---|---|---|---|---|
| 7 | GM102 20 mg/kg | 310.63 | 288 | 57.03 |
| | Cisplatin 5 mg/kg | | | |
| 8 | GM102 20 mg/kg | 315.31 | 320 | 53.30 |
| | Gemcitabine 100 | | | |
| | mg/kg | | | |

5.3. Tumor Measurement and Animal Observations

Tumor volume was evaluated by measuring tumor diameters, with a caliper, three times a week during the treatment period. The formula TV (mm³)=[length (mm)×width (mm)²]/2 was used, where the length and the width are the longest and the shortest diameters of the tumor, respectively.

All animals were weighed three times a week during the treatment period. Adverse effect of the different treatments was determined as:
Relative Body Weight (RBW) will be calculated for each measurement by dividing the body weight by the body weight at the start of treatment.
Individual Body weight loss percent (% BWL)=100−(BWₓ/BW₀×100), where BWₓ is the BW at any day during the treatment and BW₀ is the BW on the 1^{st} day of treatment.

Mice were observed every day for physical appearance, behavior and clinical changes.

All signs of illness, together with any behavioral change or reaction to treatment, were recorded for each animal.

5.4. XTS-1526 Study Design

A total of 8 groups were used as summarized in Table 9. For the groups 1 to 6, each group initially included 9 mice. For the groups 7 and 8, each group initially included 8 mice.

In group 1, vehicle was dosed at 5 ml/kg, by intravenous route twice a week for 3 weeks.

In group 2, GM102 was dosed at 20 mg/kg, by intravenous route twice a week for 3 weeks.

In group 3, docetaxel was dosed at 20 mg/kg, by intravenous route once on D0.

In group 4, GM102 was dosed at 20 mg/kg, by intravenous route twice a week for 1 or 2 weeks in combination with docetaxel at 20 mg/kg, by intravenous route once on D0.

In group 5, cisplatin was dosed at 5 mg/kg combined with gemcitabine at 100 mg/kg, both by intraperitoneal route once a week for 2 or 3 weeks.

In group 6, GM102 was dosed at 20 mg/kg, by intravenous route twice a week for 1 or 2 weeks with the combination cisplatin at 5 mg/kg and gemcitabine at 100 mg/kg, both by intraperitoneal route once a week for 1 or 2 weeks.

In group 7, GM102 was dosed at 20 mg/kg, by intravenous route twice a week for 3 weeks with cisplatin at 5 mg/kg by intraperitoneal route once a week for 3 weeks.

In group 8, GM102 was dosed at 20 mg/kg, by intravenous route twice a week for 3 weeks with gemcitabine at 100 mg/kg by intraperitoneal route once a week for 3 weeks.

All treatment doses were body weight adjusted at each injection.

TABLE 10
Dose and dose schedules in the XTS-1526 efficacy study

| | | | | | | 2^{nd} Testing Agent | | | | | | | |
|---|---|---|---|---|---|---|---|---|---|---|---|---|---|
| | | 1^{st} Testing Agent | | | | | Dose | | | 3^{rd} Testing Agent | | | |
| | | | Dose | | | | mg/ | | | | Dose | | |
| Gr. | N | Agent | mg/kg | Route | Schedule | Agent | kg | Route | Schedule | Agent | mg/kg | Route | Schedule |
| 1 | 9 | Vehicle | — | IV | 2qwk x 3 | — | — | — | — | — | — | — | — |
| 2 | 9 | GM102 | 20 | IV | 2qwk x 3 | — | — | — | — | — | — | — | — |
| 3 | 9 | — | — | — | — | Docetaxel | 20 | slow | D0 | — | — | — | — |
| | | | | | | | | IV | | | | | |
| 4 | 9 | GM102 | 20 | IV | 2qwk x1- | Docetaxel | 20 | slow | D0 | — | — | — | — |
| | | | | | 2 | | | IV | | | | | |
| 5 | 9 | — | — | — | — | Cisplatin | 5 | IP | D0, | Gemcitaine | 100 | IP | D0, |
| | | | | | | | | | D7 | | | | D7 |
| | | | | | | | | | and/ | | | | and/or |
| | | | | | | | | | or | | | | D14 |
| | | | | | | | | | DI4 | | | | |
| 6 | 9 | GM102 | 20 | IV | 2qwk xl- | Cisplatin | 5 | IP | D0 | Gemcitaine | 100 | IP | D0 and |
| | | | | | 2 | | | | and | | | | D7 |
| | | | | | | | | | D7 | | | | |
| 7 | 8 | GM102 | 20 | IV | 2qwk x 3 | Cisplatin | 5 | IP | qwk | — | — | — | — |
| | | | | | | | | | x 3 | | | | |
| 8 | 8 | GM102 | 20 | IV | 2qwk x 3 | — | — | — | — | Gemcitaine | 100 | IP | qwk x |
| | | | | | | | | | | | | | 3 |

5.5. Actions Implemented in Case of Body Weight Loss or Adverse Event

If any side effects were observed or if body weight loss ≥15% compared to the day of inclusion was observed at the day of tumor measurement and body weight monitoring (three times a week), the sponsor was informed in shortest delays from the discovering of side effects/problems.

Then, the following actions were implemented:
Treatment was stopped for the concerned animal; treatment was resumed if body weight loss <10%
DietGel Recovery® was given for the entire group in which the body weight loss was observed and the corresponding animal was weighed every day until body weight loss <10%; DietGel Recovery® addition was stopped if body weight loss <10%.

5.6. Criteria for Ethical Sacrifice

Animals were sacrificed based on the following criteria:
Body weight loss (BWL)≥20% compared to the 1st day of treatment for 48 consecutive hours (3 measurements).
General alteration of behaviour or clinical signs.
Tumor volume ≥2000 mm³.

5.7. End Points/Study Termination

Only mice reaching ethical sacrifice criteria were sacrificed at the appropriate time.

All experimental groups were ended at the end of the experimental period.

The endpoints for the experiment were:
a treatment phase of 4 weeks,
no follow-up phase.

5.8. Blood, Tumor and Tissue Sampling

5.8.1. Tumor Sampling

5.8.1.1. Tumor Sampling for FFPE

½ tumor was processed for FFPE: tumor was fixed in 10% formalin for 24 hrs and transferred in ethanol 70%, and then sent to Histalim at following address for paraffin embedding (i.e. 17 [from main study] FFPE tumor samples):

Exact sampling time and duration of formalin fixation were noted for each tumor sampling.

During the amendment 5 editing, the sponsor decided to discard the FFPE samples.

5.8.1.2. Tumor Sampling for Snap Freezing

½ tumor was processed for snap freezing: tumor was cut in 3×3×3 mm pieces and snap-frozen in liquid nitrogen, then transferred to −80 C.° for storage (i.e. 17 [from main study]+6 [from tolerability 2-group study] snap-frozen tumor samples).

Exact sampling times were noted for each tumor sampling.

5.9. Data Analysis

5.9.1. Data Processing

All raw data were recorded on appropriate forms bound in numbered registers, stored and processed by a computer system.

Day 0 was considered the first day of treatment. The days of the experiment were subsequently numbered according to this definition.

Recordings are expressed as mean±standard error of the mean (m±sem).

Mean Relative body weight curves will be obtained by plotting the mean RBW against time for each experimental group. Delta relative body weights (relative body weights of treated group compared to relative body weights of control group) will be used for statistical analysis.

Mean Body weight loss percent (% BWL)=100−(mean BWx/mean BW0x 100), where BWx is the mean BW at any day during the treatment and BW0 is the mean BW on the 1st day of treatment.

Tumor growth curves were obtained by plotting the mean tumor volume in mm³ against time for each experimental group. Delta tumor volumes (relative tumor volumes of treated group compared to relative tumor volumes of control group) were used for statistical analysis.

Individual tumor growth delays (TGD) was calculated as the time in days required for individual tumors to reach 3- to 5-fold the initial tumor volume. Median growth delay/group was calculated and reported in the tables.

The tumor growth delay index (TGDI) was calculated as the median growth delay in the treated group divided by the median growth delay in the control group.

The percentage ratio between the mean tumor volume of a treated group (T) and the mean tumor volume of the control group (C) was calculated.

Statistical analysis was done for each measurement by Mann-Whitney non parametric comparison test. Each treated group was compared with control group.

Tumor Stabilization (TS) was defined as the number of mice presenting a constant tumor size during at least 3 consecutive measurements.

Partial tumor Regression (PR) was defined as the number of mice presenting a tumor size lower than initial tumor size during at least 3 consecutive measurements.

Complete tumor Regression (CR) was defined as the number of mice presenting a 0 to 13.5 mm³ tumor size during at least 3 consecutive measurements.

Tumor Free Survivor (TFS) was defined as the number of complete tumor regressions recorded up to Group Day End.

6. Results

6.1. Tolerability Data, Clinical Observations

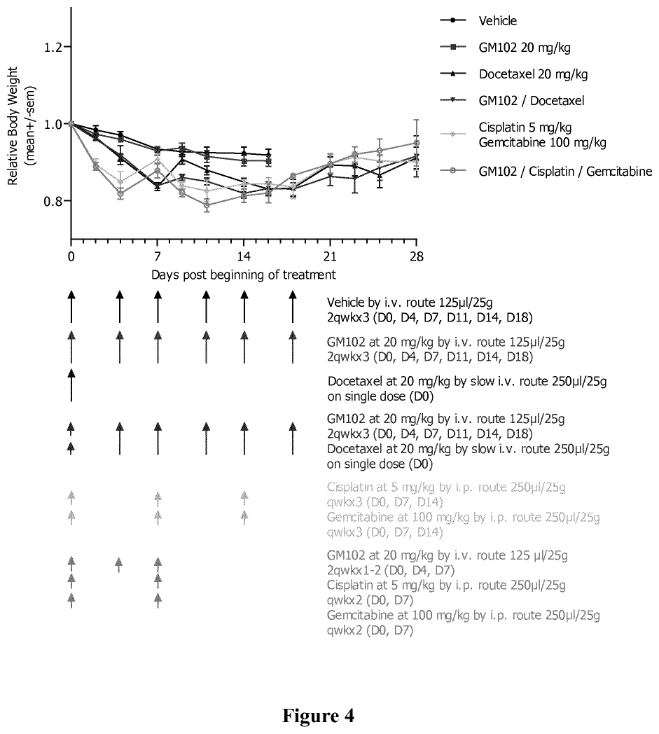

Mean percent body weight change during the treatment period is illustrated in FIG. 4.

In this study mice were weighed three times a week during the experimental period.

In group 1, vehicle dosed at 5 ml/kg, i.v. 2 qwk×3, was well tolerated but the cachectic effect of the tumor induced a maximum mean body weight loss of 8.3% on day 16 and a maximum individual body weight loss of 17.6% on day 28. No other adverse event was observed but due to cachectic effect of the tumor, DietGel Recovery® was given to the animals from the 2^{nd} inclusion on days 18, 21, 25 and 26.

In group 2, GM102 dosed at 20 mg/kg, i.v. 2 qwk×3, was well tolerated with a maximum mean body weight loss of 9.8% on day 14 and a maximum individual body weight loss of 16.8% on day 16, corresponding to the cachectic effect of the tumor as seen in control group 1. No other adverse event was observed but due to cachectic effect of the tumor, DietGel Recovery® was given to the animals from the 2^{nd} inclusion on days 11, 16, 18, from day 21 to day 27. Mouse #27 was found dead on day 27 without any clinical sign.

In group 3, docetaxel dosed at 20 mg/kg, i.v. once on D0 induced a statistically significant (p<0.01 from day 4) maximum mean body weight loss of 17.0% on day 16 compared to control group 1 and a maximum individual body weight loss of 23.8% on day 19. No other adverse event was observed but due to cachectic effect of the tumor, DietGel Recovery® was given to the whole group from day 7 to day 27 (until day 31 for animals from the 1S inclusion). Despite the given DietGel, 4 mice had to be sacrificed before the end of the study.

In group 4, GM102 dosed at 20 mg/kg, i.v. 2 qwk×1 or 2 in combination with docetaxel at 20 mg/kg, i.v. once on D0 induced a statistically significant (p<0.01 from day 4) maximum mean body weight loss of 18.1% on day 14 compared to control group 1 and a maximum individual body weight loss of 24.1% on day 23. No other adverse event was observed but due to cachectic effect of the tumor, DietGel Recovery® was given to the whole group on days 4 and 5, then from day 7 to day 27. Despite the given DietGel, 5 mice had to be sacrificed before the end of the study.

In group 5, cisplatin dosed at 5 mg/kg combined with gemcitabine at 100 mg/kg, both i.p. qwk×2 or 3 induced a statistically significant (p<0.01 from day 2) maximum mean body weight loss of 17.5% compared to control group 1 and a maximum individual body weight loss of 30.1% on day 11. Due to the combined toxicity of the compound combination and the cachectic effect of the tumor growth, DietGel Recovery® was given to the animals from the 2^{nd} inclusion on days 2 and 3, then to the whole group on days 4 and 7, then from day 9 to day 27 (until day 31 for animals from the 1^{st} inclusion). Despite the given DietGel, 4 mice had to be sacrificed before the end of the study, and 1 mouse was found dead on day 12.

In group 6, GM102 dosed at 20 mg/kg, i.v. 2 qwk×1 or 2 with the combination cisplatin at 5 mg/kg and gemcitabine at 100 mg/kg, both i.p. qwk×1 or 2 induced a significant (p<0.001 from day 2) maximum mean body weight loss of 21.1% compared to control group 1 and a maximum individual body weight loss of 27.5% on day 11. Due to the combined toxicity of the compound combination and the cachectic effect of the tumor growth, DietGel Recovery® was given to the animals from the 2^{nd} inclusion on days 2 and 3, then to the whole group from day 4 to day 27 (until day 31 for animals from the 1^{st} inclusion). Despite the given DietGel, 7 mice had to be sacrificed before the end of the study.

In additional group 7, GM102 dosed at 20 mg/kg, i.v. 2 qwk×3 with cisplatin at 5 mg/kg i.p. qwk×3, and combined with the cachectic effect of the tumor growth induced a significant maximum mean body weight loss of 12.3% on day 18 and a maximum individual body weight loss of 28.9% on day 28. Due to the combined toxicity of the compound combination and the cachectic effect of the tumor growth, DietGel Recovery® was given to the animals on days 9 and 11, then from day 13 to day 28. Despite the given DietGel, 2 mice had to be sacrificed and 1 was found dead before the end of the study. Moreover, 5/8 mice showed desquamation or/and dry skin from day 8 to the study end.

In additional group 8, GM102 dosed at 20 mg/kg, i.v. 2 qwk×3 with gemcitabine at 100 mg/kg i.p. qwk×3, and combined with the cachectic effect of the tumor growth induced a significant maximum mean body weight loss of 13.4% on day 11 and a maximum individual body weight loss of 26.4% on day 28. Due to the combined toxicity of the compound combination and the cachectic effect of the tumor growth, DietGel Recovery® was given to the animals from day 2 to day 4, from day 7 to day 9, on days 11 and 12, then from day 14 to day 28. Despite the given DietGel, 3 mice had to be sacrificed and 1 was found dead before the end of the study. Moreover, 6/8 mice showed desquamation or/and dry skin from day 4 to the study end.

6.2. Antitumor Efficacy Data

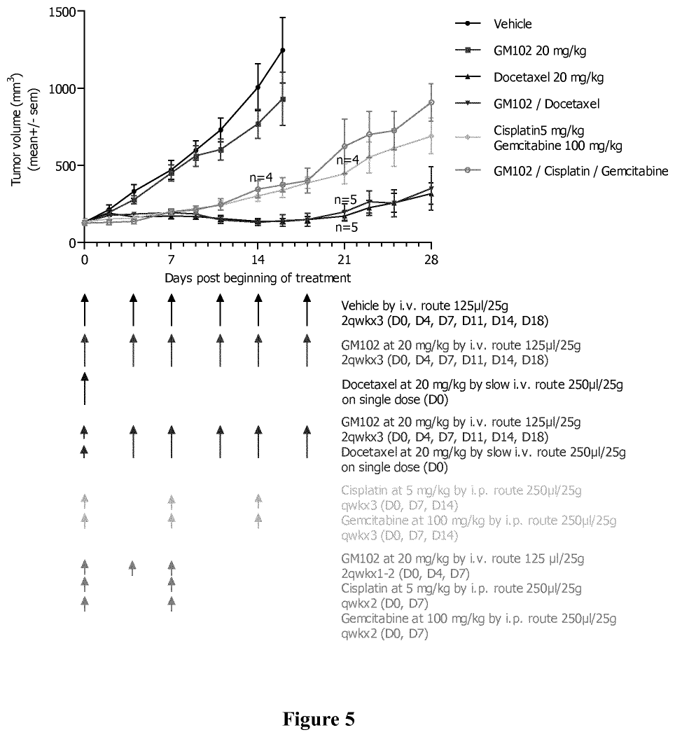

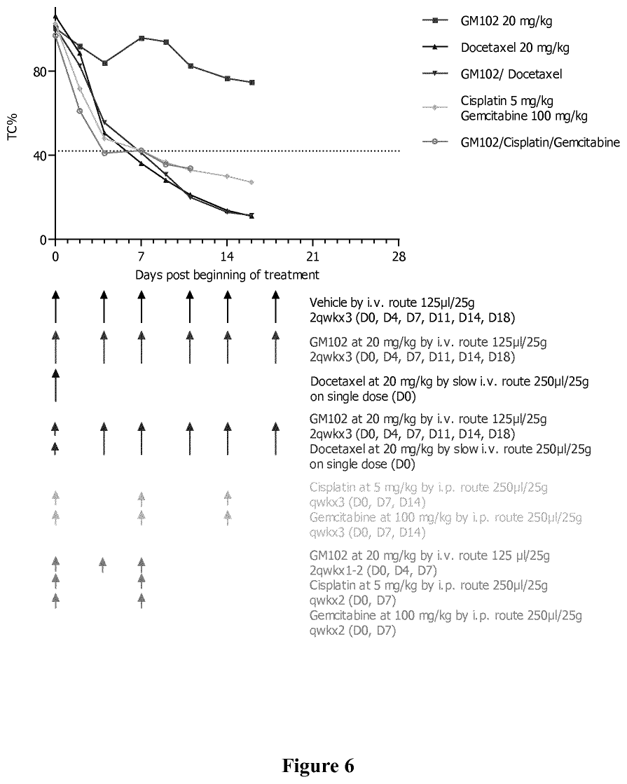

Tumor growth curves (mean tumor volume over time) are illustrated in FIG. 4. Percent T/C values for each treatment group are presented in Table 11 and illustrated in FIGS. 5 and 6. Statistical analysis is shown in Table 12.

In this study tumors were measured three times a week during the experimental period.

In group 2, GM102 dosed at 20 mg/kg, i.v. 2 qwk×3, did not demonstrate any antitumor efficacy with TGDI=1.33 and best T/C=74.68% on day 16 (end of control group).

In group 3, docetaxel dosed at 20 mg/kg, i.v. once on D0, demonstrated a strong and statistically significant (p<0.01 on D4, then p<0.001 from D7 to D16 compared with control group 1 by Man-Witney test) antitumor efficacy with TGDI>2.71 and best T/C=11.00% on day 16 (end of control group). Moreover, 7/9 transient tumor stabilizations and 2/9 transient partial tumor regressions were observed during the treatment period.

In group 4, GM102 dosed at 20 mg/kg, i.v. 2 qwk×1 or 2 in combination with docetaxel at 20 mg/kg, i.v. once on D0, demonstrated a strong and statistically significant (p<0.01 on D4, then p<0.001 from D7 to D14 compared with control group 1 by Man-Witney test) antitumor efficacy with TGDI>2.71 and best T/C=11.34% on day 16 (end of group 4, n=6). Moreover, 6/9 transient tumor stabilizations and 3/9 transient partial tumor regressions were observed during the treatment period.

In group 5, cisplatin dosed at 5 mg/kg combined with gemcitabine at 100 mg/kg, both i.p. qwk×2 or 3, demonstrated statistically significant (p<0.01 on D4, then p<0.001 from D7 to D11 compared with control group 1 by Man-Witney test) antitumor efficacy with TGDI=2.30 and best T/C=27.16% on day 16 (end of group 5, n=6). Moreover, 5/9 transient tumor stabilizations were observed during the treatment period.

In group 6, GM102 dosed at 20 mg/kg, i.v. 2 qwk×1 or 2 with the combination cisplatin at 5 mg/kg and gemcitabine at 100 mg/kg, both i.p. qwk×1 or 2, demonstrated statistically significant (p<0.05 on D2, then p<0.001 from D4 to D11 compared with control group 1 by Man-Witney test) antitumor efficacy with TGDI=1.98 and best T/C=33.71% on day 11 (end of group 6, n=7). Moreover, 6/9 transient tumor stabilizations were observed during the treatment period.

In additional groups 7 and 8, comparison with control group 1 was not possible due to higher mean tumor volume at enrolment, but several transient tumor stabilizations were observed during the treatment period, 5/8 for the combination GM102/cisplatin and 6/8 for the combination GM102/gemcitabine.

7. Conclusion

Results and Discussion

The cachectic effect of the SC131 tumor model was higher than expected and led to a similar weight loss in both vehicle and GM102-treated group. Consequently, one can consider that GM102 used alone was well tolerated.

On the other hand, the toxicity observed in the 4 other groups was partly due to the standards of care docetaxel, cisplatin and gemcitabine and induced the death of about half of the mice in each group.

The GM102 antibody used alone induced 25% of tumor growth inhibition, an effect that didn't reach statistical significance, whereas the standard of care groups showed a strong inhibition of tumor growth. This result was surprising since this model was initially selected based on its membranous AMHRII expression (scored 1+ by THC). However, when membranous AMHRII expression was evaluated on SC131 PDX tumors in parallel to this study, it was noticed that membranous AMHRII expression decreased after few passages (scored 0.2+; 40% of positive cells scored at 0.5%). This data confirmed that AMHRII expression is unstable in certain in vitro and in vivo models and that membranous expressions is critical for AMHRII antitumor efficacy.

For the same reason, no potentiation of antitumor activity was observed by combining GM102 with these standard of care.

### Example 5: In Vivo Efficacy of Anti-AMHRII Antibodies Against AMHRII-Expressing Lung Cancer

A. Materials and Methods

A.1. AMHRII Membrane Expression by Histo-Immunochemistry

A method of indirect immunofluorescence was therefore developed with the anti-AMHRII 3C23K antibody conjugated to Alexa Fluor® 488. Signal amplification was then performed in two-steps with a rabbit anti-AF488 antibody and a goat anti-rabbit antibody conjugated to Alexa Fluor® 647.

Frozen tissue sections are made with the cryostat Leica CMD1950 keep at −20° C. Frozen tissue are mounted on metal disc with OCT compound and once solidified they were mounted on the disc holder. Section of 7 μm were realized and were put on the Superfrost Plus slides (Menzel Gläser) and immediately store at −20° C.

The frozen section slides were rehydrated with PBS 1× and then fixed 10 min at −20° C. by covering them with 300 μl of cold acetone (VWR Prolabo) and recovered with parafilm to ensure that all the tissue was totally recovered by the solution. After rising with PBS, slides were treated with 300 μl of blocking buffer (PBS1×-BSA2%-Goat serum 10%-Triton X100 0.1%) 1 hour in a humidified box at RT to block unspecific interactions between antibodies and tissue components. The 3C23K-AF488 or isotype control R565-AF488 diluted at 10 μg/ml in blocking buffer were applied for 30 min at RT in the humidified box. After 3 washes with PBS1×-Triton X100 0.1% (3×10 min), antibody anti-AF488 (Invitrogen) diluted at 1/500 in blocking buffer were added (300 μl) for 30 min of incubation at RT. After 3 washes with PBS1×-Triton X100 0.1% (3×10 min), anti-rabbit antibody AF647 conjugated (Invitrogen) diluted at 1/500 in blocking buffer were added (300 μl) for 30 min of incubation at RT. Washes (3×10 min) with PBS1×-Triton X100 0.1% were realized, then DAPI (Sigma-Aldrich) at 0.5 μg/ml were applied for 10 min. After rising with PBS and H₂O the slides sections were mounted under coverslips (24×50 mm, Knittel Glass) with a drop (50 μl) of DAKO Fluorescent mouting medium avoiding bubble air and store at 4° C. in the dark until they were imaged.

Images acquisition were performed using fluorescence microscope Leica DM5000B equipped with the CoolSnap EZ CCD camera controlled by the Metavue software (Molecular Devices). Images post-treatments are performed using the ImageJ free software (http://imagej.nih.gov/ij/).

A.2. Human Lung Tumor Xenografts

Tumor fragments were obtained from xenografts in serial passage in nude mice. After removal from donor mice, tumors were cut into fragments (3-4 mm edge length) and placed in PBS containing 10% penicillin/streptomycin. Recipient animals were anesthetized by inhalation of isoflurane and received unilateral or bilateral tumor implants subcutaneously in the flank.

LXFE2226 squamous non-small cell lung cancer model tumor xenografts were implanted subcutaneously with one tumor per mouse (NMRI-Foxn1^{nu} from Charles River). The experiment consisted of two groups of mice from which three of them were euthanized on day 15 for detecting membranous AMHRII expression by flow cytometry. The first group was a vehicle control group and the second group received the investigational antibody GM102, which was administered intraperitoneally (i.p.) twice weekly at a dose level of 20 mg/kg.

Antitumor efficacy was evaluated as minimum T/C value by comparison of group median relative tumor volumes (RTVs) on the days where the optimal efficacy was reached. The experiment was terminated on day 43 after a two-week dosing-free observation period.

Study Design:

| | | Total Daily | | | |
|---|---|---|---|---|---|
| Group | | Dose | Schedule | Appl. | No. of |
| ID | Therapy | [mg/kg/day] | [Dosing days] | Route | Animals |
| 1 | PBS as Control | 10 ml/kg | BIW*4 | i.p. | 13^{#} |
| | Vehicle | | | | |
| 2 | GM102 | 20 | BIW*4 (h: 0) | i.p. | 14^{#} |

B. Results

B. In Vivo Activity of the GM 102 Anti-AMHRII Antibody Against Lung Tumors

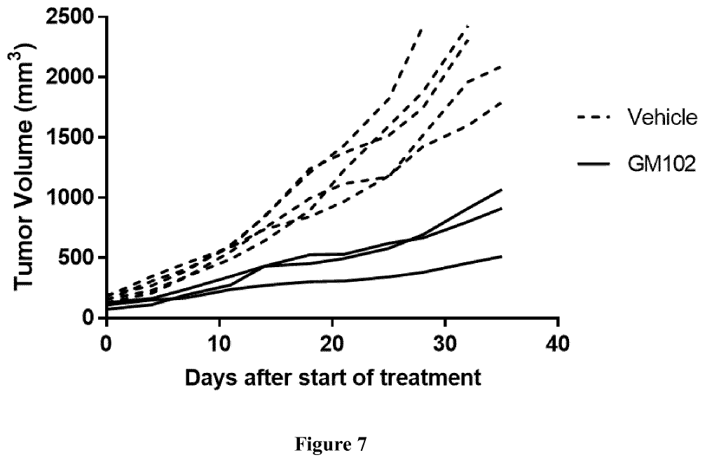

Tumor growth curves (mean tumor volume over time) are illustrated in FIG. 7. The tumors were measured three times a week during the experimental period.

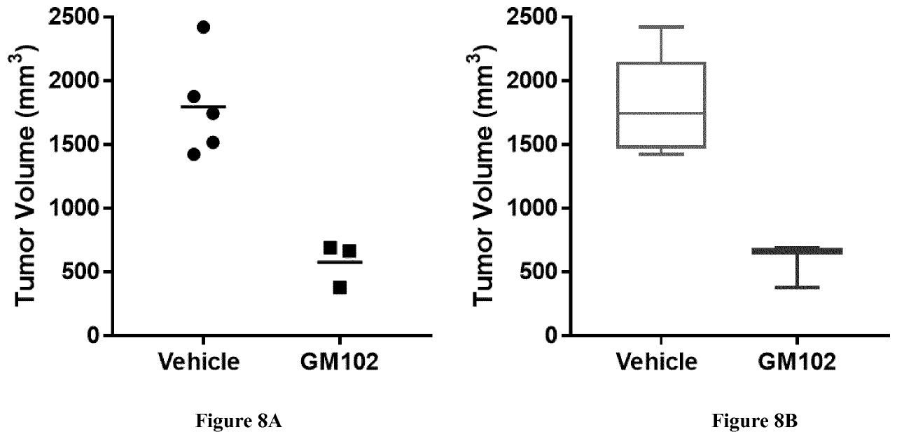

The results depicted in FIGS. 7 and 8 show that the anti-AMHRII antibody GM102 demonstrated a strong anti-tumor activity in all xenografted animals treated.

The measures of tumor growth at Day 28 illustrate that the anti-AMHRII antibody GM102 has caused a drastically reduction of the tumor volume (p<0.001), which means that the anti-AMHRII antibody (i) has prevented tumor growth and (ii) has efficiently caused the lysis of the tumor cells initially contained in the tumor xenografts.

Thus, the results of Example 5 showed that the anti-AMHRII antibody exerts a highly efficient anti-tumor effect against the lung cancer cells that actually express the AMHRII protein at their membrane, irrespective of the level of expression of the AMHRII-encoding gene.

TABLE 11
Anti-tumor activity of GM102, alone or in combination with standard of care in the SC131 xenograft, efficacy study XTS-1526

| | | Dose | | | | Dose | | | | Dose | | |
|---|---|---|---|---|---|---|---|---|---|---|---|---|
| Gr. | Drug (1) | (mg/kg) | Route | Schedule | Drug (2) | (mg/kg) | Route | Schedule | Drug (3) | (mg/kg) | Route | Schedule |
| G1 | Vehicle | — | IV | 2qwkx3 | | | | | | | | |
| G2 | GM102 | 20 | IV | 2qwkx3 | | | | | | | | |
| G3 | | | | | Docetaxel | 20 | slow IV | D0 | | | | |
| G4 | GM102 | 20 | IV | 2qwkx1-2 | Docetaxel | 20 | slow IV | D0 | | | | |
| G5 | | | | | Cisplatin | 5 | IP | qwkx2-3 | Gemcitabine | 100 | IP | qwkx2-3 |
| G6 | GM102 | 20 | IV | 2qwkx1-2 | Cisplatin | 5 | IP | qwkx1-2 | Gemcitabine | 100 | IP | qwkx1-2 |
| | Anti-tumor activity of GM102, alone or in combination with standard of care in the SC131 xenograft, efficacy study XTS-1526 | | | | | | | | | | | |
| | | Mean Tumor | median TGD | | | | | | | | | |
| | | Volume at | × 5 | | T/C % | | | | | | | Mice |
| | Gr. | D0 (mm3) | (in days) | TGDI | at day 16 | BestT/C% | at Day | TS | PR | CR | TFS | Nb |
| | G1 | 129.3 | 10.32 | / | / | / | / | 0 | 0 | 0 | 0 | 9 |
| | G2 | 129.8 | 13.70 | 1.33 | 74.68% | 74.68% | D16 | 0 | 0 | 0 | 0 | 9 |
| | G3 | 137.3 | >28 | >2.71 | 11.00% | 11.00% | D16 | 7 | 2 | 0 | 0 | 9 |
| | G4 | 130.3 | >28 | >2.71 | 11.34% | 11.34% | D16 | 6 | 3 | 0 | 0 | 9 |
| | G5 | 132.7 | 23.74 | 2.30 | 27.19% | 27.19% | D16 | 5 | 0 | 0 | 0 | 9 |
| | G6 | 125.2 | 20.41 | 1.98 | / | 33.71% | D11 | 4 | 0 | 0 | 0 | 9 |

XenTech T/C = Mean tumor volume of treated mice / Mean tumor volume of control mice xl00 (calculated at the time of first ethical sacrifice in control group);
TGD (Tumor Growth Delay) = time required for the median tumor volume to reach D0 tumor volume x5;
TGDI (Tumor Growth Delay Index) = TGD from treated/TGD from control mice;
TS (Tumor Stabilization) = number of mice presenting a constant tumor size during at least 3 consecutive measurements;
PR (partial regression) = number of mice presenting a tumor size lower than initial tumor size during at least 3 consecutive measurements;
CR (complete regression) = number of mice presenting a 0 to 13 mm³ tumor size during at least 3 consecutive measurements;
TFS (Tumor Free Survivor) = number of compete regressions recorded up to Group Day End. Treatments started on day 18 post implantation.

TABLE 12
Summary of Mann-Whitney analysis on tumor volume in the SC131 tumor model, efficacy study XTS-1526

| MANN-WHITNEY TEST | | DAY | 0 | 2 | 4 | 7 | 9 | 11 | 14 | 16 |
|---|---|---|---|---|---|---|---|---|---|---|
| Vehicle - 2qwkx3 | vs GM102 20 2qak3 | | ns | ns | ns | ns | ns | ns | ns | ns |
| Vehicle - 2qwk3 | vs Docetaxel 20 D0 | | ns | ns | ** | *** | *** | *** | *** | *** |
| Vehicle - 2qwk | vs GM102 20 2qwkx1-2 Docetaxel 20 D0 | | ns | ns | ** | *** | *** | *** | *** | |
| Vehicle - 2qwk3 | vs Cisplatin 5 qwkx2-3 Gemcitabine 100 qwkx2-3 | | ns | ns | ** | *** | *** | *** | | |
| Vehicle - 2qwkx32 | vs GM102 20 2qwkx1-2 Cisplatin 5 qwkx1-2 Gemcitabine 100 | qwlod-2 | ns | * | *** | *** | *** | *** | | |

Group comparisons were carried out using a Mann-Whitney nonparametric test between treated group and control group:
ns = not significant,
* = P < 0.05,
** = P < 0.01, and
*** = P < 0.001. Initial group size: 9 animals.

1. A method of preventing or treating a lung cancer in a patient affected with a lung cancer selected from the group consisting of epidermoid NSCLC, adenocarcinoma NSCLC, large cells NSCLC, squamous cell carcinoma NSCLC, pleiomorphic cell carcinoma NSCLC and neuroendocrine NSCLC comprising a step of administering to said patient in need thereof an AMHRII-binding agent.

2. The method according to claim 1, wherein the AMHRII-binding agent is selected in the group consisting of a monoclonal anti-AMHRII antibody and AMHRII-binding fragments thereof.

3. The method according to claim 1, wherein the AMHRII-binding agent is a monoclonal antibody selected in the group consisting of the following antibodies:
a) a light chain comprising SEQ ID NO: 2 and a heavy chain comprising SEQ ID NO: 4 (3C23 VL and VH sequences without leaders);
b) a light chain comprising SEQ ID NO: 6 and a heavy chain comprising SEQ ID NO: 8 (3C23K VL and VH sequences without leaders);
c) a light chain comprising SEQ ID NO: 10 and a heavy chain comprising SEQ ID NO: 12 (3C23 light and heavy chains without leaders);
d) a light chain comprising SEQ ID NO: 14 and a heavy chain comprising SEQ ID NO: 16 (3C23K light and heavy chains without leaders).

4. The method according to claim 1, wherein the AMHRII-binding agent is a monoclonal antibody comprising CDRs comprising the following sequences:
-CDRL-1: RASX1X2VX3X4X5A, where Xi and X2are,
independently, S or P, X3is R or W or G,
X4is T or D, and X5is I or T;
-CDRL-2 is
PTSSLX6S where X6is K or E;
and
-CDRL-3 is
LQWSSYPWT;
-CDRH-1 is
KASGYX7FTX8X9HIH where X7is S or T, X8is S or G
and X9is Y or N
-CDRH-2 is
WIYPX10DDSTKYSQKFQG where Xwis G or E; and
-CDRH-3 is
GDRFAY

5. The method according to claim 1, wherein the said AMHRII-binding agent consists of an Antibody Drug Conjugate (ADC).

6. The method according to claim 1, wherein the AMHRII-binding agent is an AMHRII-binding engineered receptor.

7. The method according to claim 1, wherein the AMHRII-binding agent is a cell expressing an AMHRII-binding engineered receptor.

8. The method according to claim 7, wherein the AMHRII-binding agent is a CAR T-cell, a CAR NK cell or a CAR Macrophage expressing an AMHRII-binding engineered receptor.

9. The method according to claim 1, wherein the said human AMHRII-binding agent is combined with one or more distinct anti-cancer agent(s).

10. A method for determining whether an individual affected with a lung cancer is responsive to a cancer treatment with an AMHRII-binding agent, wherein the method comprises the step of determining whether a tumor tissue sample previously obtained from the said individual expresses the AMHRII protein at the cell surface.


SEQUENCE LISTING (74 sequences)

SEQ ID NO: 1 - DNA, 318 bp, Artificial Sequence
  Synthetic 3C_23 VL without leader CDS 1..318
gacatccagatgacacagtccccatctaccctgtctgcttccgtgggagatcgggtgactatcacctgcagagcaagctcctccgtgaggtacatcgcttggtaccagcagaagccaggaaaggccccaaagctgctgacctacccaacctcctccctggaatccggggtgcccagcagattctcaggcagtggctccggcaccgaattcaccctgaccatcagctcactgcagcctgacgacttcgcaacctactactgtctgcagtggagtagctacccttggacattcggcggcggcaccaaggtggagatcaag

SEQ ID NO: 2 - protein, 106 aa, Artificial Sequence
  Synthetic Construct CDS1..318 from SEQ ID NO 1
DIQMTQSPSTLSASVGDRVTITCRASSSVRYIAWYQQKPGKAPKLLTYPTSSLESGVPSRFSGSGSGTEFTLTISSLQPDDFATYYCLQWSSYPWTFGGGTKVEIK

SEQ ID NO: 3 - DNA, 345 bp, Artificial Sequence
  Synthetic 3C_23 VH without leader CDS 1..345
caggtgcggctggtgcagagcggggccgaggtgaagaagcctggagcctcagtgaaggtgagttgcaaggcctccggttacaccttcaccagctaccacatccactgggtcagacaggctcccggccagagactggagtggatgggctggatctaccctggagatgactccaccaagtactcccagaagttccagggtcgcgtgaccattaccagggacaccagcgcctccactgcctacatggagctgtcttccctgagatctgaggataccgcagtctactactgtacacggggggaccgctttgcttactgggggcagggcactctggtgaccgtctcgagc

SEQ ID NO: 4 - protein, 115 aa, Artificial Sequence
  Synthetic Construct [CDS]1..345 from SEQ ID NO 3
QVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 5 - DNA, 318 bp, Artificial Sequence
  Synthetic 3C_23K VL without leader CDS 1..318
gacatccagatgacacagtccccatctaccctgtctgcttccgtgggagatcgggtgactatcacctgcagagcaagctcctccgtgaggtacatcgcttggtaccagcagaagccaggaaaggccccaaagctgctgacctacccaacctcctccctgaaatccggggtgcccagcagattctcaggcagtggctccggcaccgaattcaccctgaccatcagctcactgcagcctgacgacttcgcaacctactactgtctgcagtggagtagctacccttggacattcggcggcggcaccaaggtggagatcaag

SEQ ID NO: 6 - protein, 106 aa, Artificial Sequence
  Synthetic Construct [CDS]1..318 from SEQ ID NO 5
DIQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 7 - DNA, 345 bp, Artificial Sequence; identical to SEQ ID NO: 3
  Synthetic 3C_23K VH without leader CDS 1..345

SEQ ID NO: 8 - protein, 115 aa, Artificial Sequence; identical to SEQ ID NO: 4
  Synthetic Construct [CDS]1..345 from SEQ ID NO 7

SEQ ID NO: 9 - DNA, 639 bp, Artificial Sequence
  Synthetic 3C_23 light chain without leader CDS 1..639
gacatccagatgacacagtccccatctaccctgtctgcttccgtgggagatcgggtgactatcacctgcagagcaagctcctccgtgaggtacatcgcttggtaccagcagaagccaggaaaggccccaaagctgctgacctacccaacctcctccctggaatccggggtgcccagcagattctcaggcagtggctccggcaccgaattcaccctgaccatcagctcactgcagcctgacgacttcgcaacctactactgtctgcagtggagtagctacccttggacattcggcggcggcaccaaggtggagatcaagcggaccgtcgccgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 10 - protein, 213 aa, Artificial Sequence
  Synthetic Construct [CDS]1..639 from SEQ ID NO 9
DIQMTQSPSTLSASVGDRVTITCRASSSVRYIAWYQQKPGKAPKLLTYPTSSLESGVPSRFSGSGSGTEFTLTISSLQPDDFATYYCLQWSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1335 bp, Artificial Sequence
  Synthetic 3C_23 heavy chain without leader CDS 1..1335
caggtgcggctggtgcagagcggggccgaggtgaagaagcctggagcctcagtgaaggtgagttgcaaggcctccggttacaccttcaccagctaccacatccactgggtcagacaggctcccggccagagactggagtggatgggctggatctaccctggagatgactccaccaagtactcccagaagttccagggtcgcgtgaccattaccagggacaccagcgcctccactgcctacatggagctgtcttccctgagatctgaggataccgcagtctactactgtacacggggggaccgctttgcttactgggggcagggcactctggtgaccgtctcgagcgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 445 aa, Artificial Sequence
  Synthetic Construct [CDS]1..1335 from SEQ ID NO 11
QVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 639 bp, Artificial Sequence
  Synthetic 3C_23K light chain without leader CDS 1..639
gacatccagatgacacagtccccatctaccctgtctgcttccgtgggagatcgggtgactatcacctgcagagcaagctcctccgtgaggtacatcgcttggtaccagcagaagccaggaaaggccccaaagctgctgacctacccaacctcctccctgaaatccggggtgcccagcagattctcaggcagtggctccggcaccgaattcaccctgaccatcagctcactgcagcctgacgacttcgcaacctactactgtctgcagtggagtagctacccttggacattcggcggcggcaccaaggtggagatcaagcggaccgtcgccgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 14 - protein, 213 aa, Artificial Sequence
  Synthetic Construct CDS1..639 from SEQ ID NO 13
DIQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 1335 bp, Artificial Sequence; identical to SEQ ID NO: 11
  3C_23K heavy chain without leader CDS 1..1335

SEQ ID NO: 16 - protein, 445 aa, Artificial Sequence; identical to SEQ ID NO: 12
  Synthetic Construct [CDS]1..1335 from SEQ ID NO 15

SEQ ID NO: 17 - protein, 17 aa, Homo sapiens
  Synthetic signal peptide
MLGSLGLWALLPTAVEA

SEQ ID NO: 18 - protein, 556 aa, Homo sapiens
PPNRRTCVFFEAPGVRGSTKTLGELLDTGTELPRAIRCLYSRCCFGIWNLTQDRAQVEMQGCRDSDEPGCESLHCDPSPRAHPSPGSTLFTCSCGTDFCNANYSHLPPPGSPGTPGSQGPQAAPGESIWMALVLLGLFLLLLLLLGSIILALLQRKNYRVRGEPVPEPRPDSGRDWSVELQELPELCFSQVIREGGHAVVWAGQLQGKLVAIKAFPPRSVAQFQAERALYELPGLQHDHIVRFITASRGGPGRLLSGPLLVLELHPKGSLCHYLTQYTSDWGSSLRMALSLAQGLAFLHEERWQNGQYKPGIAHRDLSSQNVLIREDGSCAIGDLGLALVLPGLTQPPAWTPTQPQGPAAIMEAGTQRYMAPELLDKTLDLQDWGMALRRADIYSLALLLWEILSRCPDLRPDSSPPPFQLAYEAELGNTPTSDELWALAVQERRRPYIPSTWRCFATDPDGLRELLEDCWDADPEARLTAECVQQRLAALAHPQESHPFPESCPRGCPPLCPEDCTSIPAPTILPCRPQRSACHFSVQQGPCSRNPQPACTLSPV

SEQ ID NO: 19 - protein, 115 aa, Artificial Sequence; identical to SEQ ID NO: 4
  Synthetic 3C23K/3C23

SEQ ID NO: 20 - protein, 115 aa, Artificial Sequence
  Synthetic 3C23KR/6B78
QVQLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 21 - protein, 115 aa, Artificial Sequence
  Synthetic 5B42
QVQLVQSGAEVKKPGASVKVSCKASGYTFTSYHIHWVRQAPGQRLEWMGWIYPGDDSTKYSQKFQGRVTITRDASASTAYMELSSLRSEDTAVYYCTRGDRFAYWGQGTLVTVSS

SEQ ID NO: 22 - protein, 115 aa, Artificial Sequence
  Synthetic K4D-24/6C59
RVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 23 - protein, 115 aa, Artificial Sequence
  Synthetic K4D-20
QVRLVQSGAEVKKPGASVKVSCKASGYTFTSNHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 24 - protein, 115 aa, Artificial Sequence
  Synthetic K4A-12
QVRLVQSGAEVKKPGTSVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 25 - protein, 115 aa, Artificial Sequence
  Synthetic K5D05
QVRLVQSGAEVKKPGASVKVSCKASGYTFTGYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 26 - protein, 115 aa, Artificial Sequence
  Synthetic K5D-14
QVRLVQSGAEVKKPGASVKVSCKASGYSFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 27 - protein, 115 aa, Artificial Sequence
  Synthetic K4D-123
QVRLVQSGAEVKKPGASVKVSCKASGYTFTSYHIHWVRQAPGQSLEWMGWIYPGDDSTKYSQKFQGRVTITRDTSASTAYMELSSLRSEDTAVYYCTRGDRFAYWGQGTLVTVSS

SEQ ID NO: 28 - protein, 115 aa, Artificial Sequence
  Synthetic K4D-127/6C07
QVRLVQSGAEVKKPGASVKVSCKASGYTFTSYHIHWVRQAPGQRLEWMGWIYPGDDSTKYSQKFQGRVTTTRDTSASTAYMELSSLRSEDTAVYYCTRGDRFAYWGQGTLVTVSS

SEQ ID NO: 29 - protein, 115 aa, Artificial Sequence
  Synthetic 5C14
QVRLVQSGAEVKKPGASVKVSCKASGYTFTSYHIHWVRQAPGQRLEWMGWIYPGDDSTKYSQKFQGRVTFTRDTSASTAYMELSSLRSEDTAVYYCTRGDRFAYWGQGTLVTVSS

SEQ ID NO: 30 - protein, 115 aa, Artificial Sequence
  Synthetic 5C26
QVRLVQSGAEVKKPGASVKVSCKASGYTFTSYHIHWVRQAPGQRLEWMGWIYPGDDSTKYSQKFQGRMTITRDTSASTAYMELSSLRSEDTAVYYCTRGDRFAYWGQGTLVTVSS

SEQ ID NO: 31 - protein, 115 aa, Artificial Sequence
  Synthetic 5C27
QVRLVQSGAEVKKPGASVKVSCKASGYTFTSYHIHWVRQAPGQRPEWMGWIYPGDDSTKYSQKFQGRVTITRDTSASTAYMELSSLRSEDTAVYYCTRGDRFAYWGQGTLVTVSS

SEQ ID NO: 32 - protein, 115 aa, Artificial Sequence
  Synthetic 5C60
QVRLVQSGAKVRKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 33 - protein, 115 aa, Artificial Sequence
  Synthetic 6C13
QVRLVQSGAEVKKPGASVKVSCKASGYTFTSYHIHWVRQAPGQRLEWMGWIYPEDDSTKYSQKFQGRVTITRDTSASTAYMELSSLRSEDTAVYYCTRGDRFAYWGQGTLVTVSS

SEQ ID NO: 34 - protein, 115 aa, Artificial Sequence
  Synthetic 6C18
QVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCARGDRFAYWGQGTLVTVSS

SEQ ID NO: 35 - protein, 115 aa, Artificial Sequence
  Synthetic 6C54
QVRLVQSGAEVKKPGASVKVSCKASGYTFTSYHIHWVRQAPGQRLEWMGWIYPGDDSTKYSQKFQGRVTITRDTSASTAYMELSSLRPEDTAVYYCTRGDRFAYWGQGTLVTVSS

SEQ ID NO: 36 - protein, 106 aa, Artificial Sequence; identical to SEQ ID NO: 6
  Synthetic 3C23K

SEQ ID NO: 37 - protein, 106 aa, Artificial Sequence; identical to SEQ ID NO: 2
  Synthetic L-K55E

SEQ ID NO: 38 - protein, 106 aa, Artificial Sequence
  Synthetic L-T48I, L-P50S
DIQMTQSPSTLSASVGDRVTITCRASSSVRYIAWYQQKPGKAPKLLIYSTSSLKSGVPSRFSGSGSGTEFTLTISSLQPDDFATYYCLQWSSYPWTFGGGTKVEIK

SEQ ID NO: 39 - protein, 106 aa, Artificial Sequence
  Synthetic LT48I, L-K55E
DIQMTQSPSTLSASVGDRVTITCRASSSVRYIAWYQQKPGKAPKLLIYPTSSLESGVPSRFSGSGSGTEFTLTISSLQPDDFATYYCLQWSSYPWTFGGGTKVEIK

SEQ ID NO: 40 - protein, 106 aa, Artificial Sequence
  Synthetic LS27P, L-S28P
DIQMTQSPSTLSASVGDRVTITCRASPPVRYIAWYQQKPGKAPKLLTYPTSSLKSGVPSRFSGSGSGTEFTLTISSLQPDDFATYYCLQWSSYPWTFGGGTKVEIK

SEQ ID NO: 41 - protein, 106 aa, Artificial Sequence
  Synthetic L-M4L, L-T20A
DIQLTQSPSTLSASVGDRVAITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 42 - protein, 106 aa, Artificial Sequence
  Synthetic L-S27P
DIQMTQSPSTLSASVGDRVTITCRASPSVRYIAWYQQKPGKAPKLLTYPTSSLKSGVPSRFSGSGSGTEFTLTISSLQPDDFATYYCLQWSSYPWTFGGGTKVEIK

SEQ ID NO: 43 - protein, 106 aa, Artificial Sequence
  Synthetic L-M4L, L-S9P, L-R31W
DIQLTQSPPTLSASVGDRVTITCRASSSVWYIAWYQQKPGKAPKLLTYPTSSLKSGVPSRFSGSGSGTEFTLTISSLQPDDFATYYCLQWSSYPWTFGGGTKVEIK

SEQ ID NO: 44 - protein, 106 aa, Artificial Sequence
  Synthetic L-M4L
DIQLTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 45 - protein, 106 aa, Artificial Sequence
  Synthetic L-I33T
DIQMTQSPSTLSASVGDRVTITCRASSSVRYTAWYQQKPGKAPKLLTYPTSSLKSGVPSRFSGSGSGTEFTLTISSLQPDDFATYYCLQWSSYPWTFGGGTKVEIK

SEQ ID NO: 46 - protein, 106 aa, Artificial Sequence
  Synthetic L-M4L, L-K39E
DIQLTQSPSTLSASVGDRVTITCRASSSVRYIAWYQQEPGKAPKLLTYPTSSLKSGVPSRFSGSGSGTEFTLTISSLQPDDFATYYCLQWSSYPWTFGGGTKVEIK

SEQ ID NO: 47 - protein, 106 aa, Artificial Sequence
  Synthetic L-T22P
DIQMTQSPSTLSASVGDRVTIPCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 48 - protein, 106 aa, Artificial Sequence
  Synthetic L-Y32D
DIQMTQSPSTLSASVGDRVTITCRASSSVRDIAWYQQKPGKAPKLLTYPTSSLKSGVPSRFSGSGSGTEFTLTISSLQPDDFATYYCLQWSSYPWTFGGGTKVEIK

SEQ ID NO: 49 - protein, 106 aa, Artificial Sequence
  Synthetic L-Q37H
DIQMTQSPSTLSASVGDRVTITCRASSSVRYIAWYHQKPGKAPKLLTYPTSSLKSGVPSRFSGSGSGTEFTLTISSLQPDDFATYYCLQWSSYPWTFGGGTKVEIK

SEQ ID NO: 50 - protein, 106 aa, Artificial Sequence
  Synthetic L-G97S
DIQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFSGGTKVEIK

SEQ ID NO: 51 - protein, 106 aa, Artificial Sequence
  Synthetic L-S12P
DIQMTQSPSTLPASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 52 - protein, 106 aa, Artificial Sequence
  Synthetic L-19A
DIQMTQSPSTLSASVGDRATITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 53 - protein, 106 aa, Artificial Sequence
  Synthetic L-T72A
DIQMTQSPSTLSASVGDRVTITCRASSSVRYIAWYQQKPGKAPKLLTYPTSSLKSGVPSRFSGSGSGTEFALTISSLQPDDFATYYCLQWSSYPWTFGGGTKVEIK

SEQ ID NO: 54 - protein, 106 aa, Artificial Sequence
  Synthetic L-R31W
DIQMTQSPSTLSASVGDRVTITCRASSSVWYIAWYQQKPGKAPKLLTYPTSSLKSGVPSRFSGSGSGTEFTLTISSLQPDDFATYYCLQWSSYPWTFGGGTKVEIK

SEQ ID NO: 55 - protein, 106 aa, Artificial Sequence
  Synthetic L-M4L, L-M39K
DIQLTQSPSTLSASVGDRVTITCRASSSVRYIAWYQQMPGKAPKLLTYPTSSLKSGVPSRFSGSGSGTEFTLTISSLQPDDFATYYCLQWSSYPWTFGGGTKVEIK

SEQ ID NO: 56 - protein, 106 aa, Artificial Sequence
  Synthetic L-I2N
DNQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 57 - protein, 106 aa, Artificial Sequence
  Synthetic L-G63C, L-W91C
DIQMTQSPSTLSASVGDRVTITCRASSSVRYIAWYQQKPGKAPKLLTYPTSSLKSGVPSRFSCSGSGTEFTLTISSLQPDDFATYYCLQCSSYPWTFGGGTKVEIK

SEQ ID NO: 58 - protein, 106 aa, Artificial Sequence
  Synthetic L-R31G
DIQMTQSPSTLSASVGDRVTITCRASSSVGYIAWYQQKPGKAPKLLTYPTSSLKSGVPSRFSGSGSGTEFTLTISSLQPDDFATYYCLQWSSYPWTFGGGTKVEIK

SEQ ID NO: 59 - protein, 106 aa, Artificial Sequence
  Synthetic L-I75F
DIQMTQSPSTLSASVGDRVTITCRASSSVRYIAWYQQKPGKAPKLLTYPTSSLKSGVPSRFSGSGSGTEFTLTFSSLQPDDFATYYCLQWSSYPWTFGGGTKVEIK

SEQ ID NO: 60 - protein, 106 aa, Artificial Sequence
  Synthetic L-I2T
DTQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 61 - protein, 106 aa, Artificial Sequence
  Synthetic L-I2T, L-K42R
DTQMTQSPSTLSASVGDRVTITCRASSSVRYIAWYQQKPGRAPKLLTYPTSSLKSGVPSRFSGSGSGTEFTLTISSLQPDDFATYYCLQWSSYPWTFGGGTKVEIK

SEQ ID NO: 62 - protein, 106 aa, Artificial Sequence
  Synthetic L-Y49H
DIQMTQSPSTLSASVGDRVTITCRASSSVRYIAWYQQKPGKAPKLLTHPTSSLKSGVPSRFSGSGSGTEFTLTISSLQPDDFATYYCLQWSSYPWTFGGGTKVEIK

SEQ ID NO: 63 - protein, 106 aa, Artificial Sequence
  Synthetic L-M4L, L-T20S, L-K39E
DIQLTQSPSTLSASVGDRVSITCRASSSVRYIAWYQQEPGKAPKLLTYPTSSLKSGVPSRFSGSGSGTEFTLTISSLQPDDFATYYCLQWSSYPWTFGGGTKVEIK

SEQ ID NO: 64 - protein, 106 aa, Artificial Sequence
  Synthetic L-T69P
DIQMTQSPSTLSASVGDRVTITCRASSSVRYIAWYQQKPGKAPKLLTYPTSSLKSGVPSRFSGSGSGPEFTLTISSLQPDDFATYYCLQWSSYPWTFGGGTKVEIK

SEQ ID NO: 65 - protein, 10 aa, Artificial Sequence
  Synthetic CDRL-1 of anti-AMHRII antibodies
  VARIANT 4: Xaa in position 4 is S or P
  VARIANT 5: Xaa in position 5 is S or P
  VARIANT 7: Xaa in position 7 is R or W or G
  VARIANT 8: Xaa in position 8 is T or D
  VARIANT 9: Xaa in position 9 is I or T
RASXXVXXXA

SEQ ID NO: 66 - protein, 7 aa, Artificial Sequence
  Synthetic CDRL-2 of anti-AMHRII antibodies
  VARIANT 6: Xaa in position 6 is K or E
PTSSLXS

SEQ ID NO: 67 - protein, 9 aa, Artificial Sequence
  Synthetic CDRL-3 of anti-AMHRII antibodies
LQWSSYPWT

SEQ ID NO: 68 - protein, 13 aa, Artificial Sequence
  Synthetic CDRH-1 of anti-AMHRII antibodies
  VARIANT 6: Xaa in position 6 is S or T
  VARIANT 9: Xaa in position 9 is S or G
  VARIANT 10: Xaa in position 10 is Y or N
KASGYXFTXXHIH

SEQ ID NO: 69 - protein, 17 aa, Artificial Sequence
  Synthetic CDRH-2 of anti-AMHRII antibodies
  VARIANT 5: Xaa in position 5 is G or E
WIYPXDDSTKYSQKFQG

SEQ ID NO: 70 - protein, 6 aa, Artificial Sequence
  Synthetic CDRH-3 of anti-AMHRII antibodies
GDRFAY

SEQ ID NO: 71 - DNA, 20 bp, Artificial Sequence
  Synthetic Forward primer of AMHR2
tctggatggcactggtgctg

SEQ ID NO: 72 - DNA, 20 bp, Artificial Sequence
  Synthetic Reverse primer of AMHR2
agcagggccaagatgatgct

SEQ ID NO: 73 - DNA, 21 bp, Artificial Sequence
  Synthetic Forward primer of TBP
tgcacaggagccaagagtgaa

SEQ ID NO: 74 - DNA, 20 bp, Artificial Sequence
  Synthetic Reverse primer of TBP
cacatcacagctccccacca